(19) United States
(12) Patent Application Publication
(10) Pub. No.: US 2014/0171436 A1
(43) Pub. Date: Jun. 19, 2014

(54) TREATMENT OF ANTIFOLATE NEUROTOXICITY

(71) Applicant: Syntrix Biosystems, Inc., Auburn, WA (US)

(72) Inventors: Barton Aron Kamen, New Brunswick, NJ (US); John Anthony Zebala, Sammamish, WA (US)

(73) Assignee: Syntrix Biosystems, Inc., Auburn, WA (US)

(21) Appl. No.: 13/905,044

(22) Filed: May 29, 2013

Related U.S. Application Data
(63) Continuation of application No. 10/878,733, filed on Jun. 28, 2004, now abandoned.
(60) Provisional application No. 60/483,313, filed on Jun. 27, 2003.

(51) Int. Cl. A61K 31/485; A61K 31/519; A61J 1/03; A61K 31/13

(52) U.S. Cl. CPC A61K 31/485; A61K 31/13; A61K 31/519; A61J 1/035
USPC 514/249; 514/289; 514/662; 206/531

(57) ABSTRACT

The present invention is a method of treating antifolate neurotoxicity in a mammal suffering from or at risk of developing antifolate neurotoxicity, which comprises administering to the mammal a therapeutically effective amount of an NMDA antagonist, or a pharmaceutically acceptable salt thereof.

The application claims the benefit of U.S. Provisional Patent application No. 60/483,313 filed Jun. 27, 2003, currently pending.

### FIELD OF THE INVENTION

The present invention relates to methods and pharmaceutical compositions for treating or preventing antifolate neurotoxicity by administering to a mammal a therapeutically effective dose of a compound that directly or indirectly blocks the activity of the N-methyl-D-aspartate (NMDA) receptor.

### BACKGROUND OF THE INVENTION

A. Neuronal Injury and Treatment: The Role of the NMDA Receptor

Glutamate is the major excitatory neurotransmitter in the mammalian brain, and its interaction with specific membrane receptors is responsible for many neurologic functions, including cognition, memory, movement and sensation (Lipton and Rosenberg, N. Eng. J. Med. 330(9):613, 1994). However, in a variety of pathologic conditions, including stroke and various neurodegenerative disorders, excessive activation of glutamate receptors by glutamate and other amino acids may mediate neuronal injury or death via a mechanism that has been termed “excitotoxicity”. This form of injury appears to be predominantly mediated by excessive influx of calcium into neurons through ionic channels and mobilization from intracellular stores, triggered predominantly by activation of the glutamate receptors known as N-methyl-D-aspartate (NMDA) receptors.

In an effort to prevent this type of injury, numerous agents have been put forth as potential therapeutics to antagonize the NMDA receptor either directly, or indirectly at upstream and downstream targets, in order to treat acute and chronic neurologic disorders such ischemia, hypoxia, hypoglycemia, epilepsy, Huntington's disease, Alzheimer's disease, and amyotrophic lateral sclerosis (Lipton and Rosenberg, N. Eng. J. Med. 330(9):613, 1994). Many high-affinity antagonists originally developed in the art to treat excitotoxicity blocked virtually all NMDA receptor activity, producing unacceptable clinical side effects that included hallucinations, drowsiness and coma. Accordingly, it is now appreciated that clinically tolerated neuroprotective NMDA receptor antagonists are low-affinity, open-channel blockers with relatively fast off-rates that substantially preserve non-pathologic NMDA receptor activity (Lipton and Chen, Cell Death and Differentiation. 11:18, 2004).

Low-affinity NMDA receptor antagonists that the art has suggested as possible neuroprotective agents included dextromethorphan (Kᵢ=7 μM), dextrorphan (Kᵢ=0.9 μM), memantine (Kᵢ=0.5 μM), amantadine, and rimantadine (U.S. Pat. Nos. 4,806,543 and 5,614,560; Kornhuber and Quack, Neurosci. Lett. 195:137, 1995). Memantine and dextrophmethorphan have indeed shown efficacy in animal models of hypoxia, ischemia and epilepsy, and memantine is now approved in the United States for use in humans with moderate to severe Alzheimer's disease (Steinberg, et al., Neurol. Res. 15:174, 1993; Steinberg, et al., J. Cereb. Blood Flow Metab. 11:1015, 1991; Ferkany, et al., Eur. J. Pharmacol. 151:151, 1988; Sagratella, Pharmacol. Res. 32(1/2):1, 1995; and Tariot et al., JAMA. 291(3):317, 2004).

In spite of the potentially desirable neuroprotective activity of dextromethorphan, dextromethorphan is commonly believed unsuitable as a neuroprotectant, since very little dextromethorphan is capable of reaching the central nervous system (CNS) because of its extensive first-pass elimination in humans (Vetticaden et al., Pharmaceut. Res. 6:13-19, 1989; Ramachander et al., J. Pharm. Sci. 66:1047-1048, 1977; and U.S. Pat. No. 5,166,207). This rapid clearance makes it particularly difficult to attain the micromolar CNS concentrations necessary to fully bind the relatively weak affinity NMDA receptor. Indeed, reaching neuroprotective concentrations has been shown to require the administration of frequent and massive doses of dextromethorphan (or its metabolite dextrorphan) as large as 3300 mg/day (Walker and Hunt, Clin. Neuropharmacol. 12:322-330, 1989; Albers et al., Clin. Neuropharmacol. 15(6):509, 1992; Albers et al. Stroke. 26:254, 1995; and Steinberg et al., J. Neurosurg. 84:860, 1996). Such doses were found to produce unacceptable PCP-like side-effects that included nystagmus, nausea and vomiting, distorted vision, feeling “drunk”, ataxia and dizziness. Accordingly, dextromethorphan and dextrorphan are considered undesirable in their utility for treating NMDA-mediated neuronal injury since they require dosages that produce unacceptable side-effects in order to overcome their weak affinities for the NMDA receptor and short plasma half lives.

B. Intracellular Sigma-1 Receptors: Modulators of Signal Transduction

Sigma-1 receptors reside in the cell primarily at the endoplasmic reticulum, and in the body are distributed mainly in the CNS, but also exist in the periphery (Su and Hayashi, Curr. Med. Chem. 10:2073, 2003). Sigma-1 receptors bind to numerous ligands, including (+)-benzomorphans like (+)-pentazocine, (+)N-allyl-normetazocine, dextromethorphan and dextrorphan. In contrast to NMDA receptor binding by dextromethorphan and dextrorphan, the high affinity of sigma-1 receptors for (+)-benzomorphinans (Kᵢs of 100-200 nM) permits dextromethorphan and its metabolite dextrorphan to efficiently bind sigma-1 CNS receptors using doses that are without side-effects, in spite of their rapid first-pass elimination (Steinberg, et al., J. Neurosurg. 84:860, 1996; East and Dye, J. Chromatogr. 338:99, 1985; Barnhart, Toxicol. Appl. Pharmacol. 55:43, 1980).

Many pharmacological and physiological actions have been attributed to sigma-1 receptors. These include the regulation of 1P3 receptors and calcium signaling at the endoplasmic reticulum, mobilization of cytoskeletal adaptor proteins, modulation of nerve growth factor-induced neurite sprouting, modulation of neurotransmitter release and neuronal firing, modulation of potassium channels as a regulatory subunit, alteration of psychostimulant-induced gene expression, and blockade of spreading depression. Behaviorally, sigma-1 receptors are involved in learning and memory, psychostimulant-induced sensitization, cocaine-induced conditioned place preference, and pain perception. Notably, in almost all the aforementioned biochemical and behavioral tests, sigma-1 agonists, while having no effects by themselves, caused the amplification of signal transductions incurred upon the stimulation of the glutamatergic, dopaminergic, IP3-related metabotropic, or nerve growth factor-related systems.

Accordingly, sigma-1 receptors have been hypothesized to act as intracellular modulators, or ‘volume controls’ of signal transduction (Su and Hayashi, Curr. Med Chem. 10:2073, 2003 and Hayashi and Su, CNS Drugs. 18(5):269, 2004). In particular, studies have shown that low- and high-affinity sigma-1 ligands including amantadine (Kᵢ=7.4 μM), memantine (Kᵢ=2.6 μM), dextromethorphan (Kᵢ=0.205 μM) and dextrorphan (Kᵢ=0.144 μM) bind to sigma-1 receptors and modulate the activity of a variety of receptors including, for example, the dopamine, bradykinin and opioid receptors (Peeters, et al., Eur. J. Neurosci. 19:2212, 2004; Hayashi and Su, Proc. Natl. Acad. Sci. U.S.A. 98(2):491, 2001; Allen, et al., J. Pharmacol. Exp. Ther. 300:435, 2002).

With respect to NMDA receptors, cell-based and electrophysiological studies have both supported (Monnet, et al., Eur. J. Pharmacol. 179(3):441, 1990; Hayashi et al., J. Pharmacol. Exp. Ther. 275(1):207, 1995; Chaki, et al., Neurochem. Int. 33(1):29, 1998; Gronier and Debonnel, Eur. J. Pharmacol. 368(2-3):183, 1999; Karasawa, et al., Life Sci. 70(14):1631, 2002; Wang and Takigawa, Int. J. Neuropsychopharmacol. 5(3):239, 2002; and Martin, et al., Brain Res. Mol. Brain Res. 123(1-2):66, 2004), and contradicted the role of the sigma-1 receptor in modulating NMDA receptor activity (Fletcher, et al., Br. Pharmacol. 116(7):2791, 1995; Thurgur and Church, Br. J. Pharmacol. 124(5):917, 1998; Lynch and Gallagher, J. Pharmacol. Exp. Ther. 279(1):154, 1996; Whittemore et al., J. Pharmacol. Exp. Ther. 282(1):326, 1997; and Nishikawa, et al., Eur. J. Pharmacol. 404(1-2):41, 2000). In most cases where studies have contradicted the role of the sigma-1 receptor in modulating NMDA receptor activity, investigators have suggested that modulation of NMDA receptor was due to direct binding and inhibition of the NMDA receptor by a sigma-1 ligand used at a non-selective concentration, rather than by binding to the sigma-1 receptor per se.

Still other studies have suggested that the ability of sigma-1 receptors to provide neuroprotection depends on the type or magnitude of neuronal injury, possibly by preventing the release of excitotoxic neurotransmitters rather than indirectly modulating the NMDA receptor (Lockhart, et al., Brain Res. 675(1-2):110, 1995; and Nakazawa, et al. Neurochem. Int. 32(4):337, 1998). The prior art is therefore unclear under what conditions sigma-1 binding can indirectly modulate NMDA receptor activity either in vitro or in a mammal, or whether dextromethorphan or dextrorphan can be used therapeutically at sigma-1 selective dosages to therapeutically treat a human condition.

C. Antifolate Neurotoxicity

Methotrexate (MTX), or amethopterin, is a potent antifolate that was introduced in clinical practice more the 50 years ago. MTX is a major component of therapeutic regimens used to treat patients with osteogenic sarcoma (OS), acute lymphoblastic leukemia (ALL), medulloblastoma, head and neck cancer, non-Hodgkin's lymphoma (NHL), neoplastic meningitis, and primary CNS lymphoma. It is also used to treat patients with several non-neoplastic conditions including psoriasis, rheumatoid arthritis, juvenile idiopathic arthritis, and systemic lupus erythematosus. Other antifolates in the same therapeutic and structural class as MTX include, but are not limited to, aminopterin, trimetrexate, edatrexate, raltritrexed and lometrexol (see Kamen, Semin. Oncol. 24(5):S18, 1997).

Although MTX is considered to have an acceptable toxicity profile, the development of antifolate-mediated neurotoxicity (AF-NT) is recognized as a serious complication of the drug that can result in neurologic deficits that range from mild and transient behavioral changes and memory loss to permanent coma and even death (see reviews by Ochs, Am. J. Pediatr. Hematol. Oncol. 11:93, 1989 and Vesmar, et al., Chemotherapy. 49:92, 2003). While not as thoroughly characterized in patients as MTX, other antifolates in the same therapeutic and structural class would be expected to present with a substantially identical or overlapping toxicology profile as MTX. As defined for MTX (see Table I), the clinical presentation of AF-NT has been classified into acute, subacute and delayed (chronic) syndromes according to the time of appearance after initiating therapy and associated findings (see Quinn and Kamen, J. Invet. Med. 44(9):522, 1996 and Muchi, et al., Jpn. J. Clin. Oncol. 12:363, 1982).

The acute syndrome begins immediately or within hours of MTX infusion and can result in somnolence, confusion, fatigue, disorientation and seizures (see Bleyer, Cancer Treat. Rep. 65 Suppl. 1:89, 1981). A clinical picture of chemical arachnoiditis may also appear, comprising symptoms of headache, nausea, vomiting, fever, back pain, and dizziness. The duration of symptoms are usually 12-72 hours (see Weiss, et al., N. Engl. J. Med. 291:127, 1974).

The subacute syndrome is typically associated with high-dose MTX and presents days to weeks following exposure to MTX with an encephalopathy that is characterized by hemiparesis, ataxia, speech disorders, seizures, confusion, and affective disturbances (see Packer et al., Med. Pediatr. Oncol. 11:159, 1983; Walker, et al., Proc. Am. Soc. Clin. Oncol. 20:84, 1984; Walker, et al., J. Clin. Oncol. 4:1845, 1986; Jaffe, et al., Cancer 56:1356, 1985; Nigro et al., Med. Pediatr. Oncol. 35:449, 2000; Winick et al., J. Natl. Cancer Inst. 84:252, 1992; Maytal et al., Epilepsia 36:831, 1995; Ochs et al., Lancet 2:1422, 1984; Yim et al., Cancer 67:2058, 1991; and Fritsch and Urban, Cancer 53:1849, 1984). Radiographic findings in the CNS are typically absent, and subsequent MTX courses do not have an increased risk of recurrence. The mean duration of symptoms have been reported to be approximately 40 hours, with a duration that ranged from 10 minutes to 240 hours (see Jaffe, et al., Cancer 56:1356, 1985 and Walker, et al., J. Clin. Oncol. 4:1845, 1986).

TABLE I
The terms Low, Moderate, and High refer to the indicated dose levels of MTX.

| | Acute Syndrome | Subacute Syndrome | Delayed (chronic) Syndrome |
|---|---|---|---|
| Typical MTX | >1 g/m², IT | 8-12.5 g/m², IV weekly. | Low - <10 mg/m² oral. |
| dose | | Moderate - 10-1000 mg/m² IT. | Moderate - 10-1000 mg/m², oral, IT, IV. |
| | | | High - 7-20 g/m² every 1-2 weeks. |
| Typical time of | Immediate to within | Days to weeks. | Low - Unknown. |
| onset from start | a day. | | Moderate - Weeks to months. |
| of MTX therapy | | | High - Months to years. |
| Incidence | 5-40%. | 4%-15% | Low - 56%. |
| | | | Moderate - 1.9-19.5% depending on net dose. |
| | | | High - 2.3%. |
| Typical findings | meningismus, | behavior abnormalities, focal | Low - memory, lethargy, headache. |
| | headache, seizures, | sensorimotor signs (mono- or | Moderate - Seizures, parathesias, paresis, |
| | vomiting, chemical | hemiparesis with aphasia), and | ataxia and headaches. |
| | arachnoiditis, fever, | abnormal reflexes, hemiplegia with | High - Behavioral to lethargy, progressive |
| | CSF pleocytosis. | a speech disorder, seizures. | dementia, seizures, spasticity and stupor. |
| Typical duration | 12-72 hours. | Mean 38 hours. | Low - Unknown. |
| of symptoms | | | Moderate - 1-2 weeks if MTX stopped. |
| | | | High - Insidiously progressive over 3-6 months. |
| Permanent | May result. | Typically none. | Low - Rare if at all. |
| sequelae | | | Moderate - Recurrent seizures 7-26%, |
| | | | neuropsychologic impairment 15%, |
| | | | asymptomatic in 63-74%. |
| | | | High - Mostly yes. Occasionally some |
| | | | symptoms may regress slightly. |
| Tendency to recur | Yes. | Usually does not. When it does, | Not applicable. |
| with more MTX | | invariably more severe. | |
| Radio-graphic | | Substantially none. | Low - Unknown, but probably rare. |
| findings | | | Moderate - Leukoencephalopathy in 15-75%, |
| | | | depending on dose. |
| | | | High - All with diffuse white matter |
| | | | hypodensity and atrophic changes. |

The delayed syndrome may develop months to years following low-, moderate-, and high-dose MTX (see Allen, et al., Cancer Treat. Rep. 64:1261, 1980; Walker, et al., J. Clin. Oncol. 4:1845, 1986; Fritsch and Urban, Cancer 53:1849, 1984; Mahoney, et al., J. Clin. Oncol. 16(5):1712, 1998; Bettachi, et al., Arthritis Rheum. 42:S236, 1999; Schagen et al., Cancer 85:640, 1999; Butler et al., J. Clin. Oncol. 12:2621, 1994; and Gay, et al., J. Child Neurol. 4:207, 1989.) Symptoms can range from memory deficits, lethargy, sexual dysfunction, insomnia, confusion, agitation, and headache in low doses to headache, seizures, parathesias, paresis, and ataxia in moderate doses. In high doses, progressive dementia, spasticity, stupor and even death may result. As the net dose of MTX escalates, the tendency of neurologic deficits to progress and become permanent over the course of several months increases. Progression and permanent sequelae occur even upon cessation of MTX. Radiographic and pathologic findings in a subpopulation of delayed syndrome patients are a characteristic feature that increases in frequency and severity in proportion to the net dose of MTX. These findings can comprise demyelination, multifocal white matter necrosis, astrocytosis, intracerebral calcifications, cerebral atrophy and mineralising microangiopathy (see Kaplan and Wiernik, Semin. Oncol. 9:103, 1982; Flament-Durand, et al., Cancer 35:319, 1975; McIntosh et al., J. Pediatr. 91:909, 1977; Peylan-Ramu, et al., N. Engl. J. Med. 298:815, 1978; Colosimo, et al., Rays 19:511, 1994; and Price and Jamieson, Cancer 35:306, 1975). In patients exposed to moderate doses of MTX and in whom symptoms reversed, a duration of symptoms of 1-2 weeks has been reported (see Gay, et al., J. Child Neurol. 4:207, 1989).

The exact pathophysiological mechanisms of AF-NT are still not understood, and extensive scientific speculation over the last two decades has resulted in well over a dozen hypotheses aimed at explaining the origin of AF-NT. Investigators have suggested that AF-NT may be the result of: (1) antifolate-mediated astrocytosis and a subsequent axonopathy, (2) ischemia secondary to tumor microemboli, (3) ischemia secondary to an alteration of the cerebral vasculature, (4) transient vasospasm, (5) altered levels of homovanillic acid and 5-hydroxyindoleacetic acid, (6) altered regional glucose metabolism, (7) toxic oxidized folates, (8) deficient tetrahydrobiopterin, (9) endothelial and microglial death resulting in accumulations of toxic metabolites, (10) an excess of excitatory amino acids (e.g. glutamate, aspartate, homocysteic acid, cysteine sulfinic acid), (11) an excess of homocysteine (possibly also acting as an excitatory amino acid), (12) an excess of adenosine, (13) an excess of S-adenosyl homocysteine resulting in a relative deficiency in S-adenosyl methionine and resultant demyelination, and (14) a chronic depletion of folates (see Vesmar et al., Chemotherapy. 49:92, 2003; Bettachi, et al., Arthritis Rheum. 42:S236, 1999; Quinn and Kamen, J. Invet. Med. 44(9):522, 1996; Quinn et al., J. Clin. Oncol. 15(8):2800, 1997; Quinn et al., J. Pediatr. Hematol. Oncol. 26:386, 2004; Walker, et al., Proc. Am. Soc. Clin. Oncol. 20:84, 1984; Walker, et al., J. Clin. Oncol. 4:1845, 1986; Jaffe, et al., Cancer 56:1356, 1985; Allen, et al., Cancer Treat. Rep. 64:1261, 1980; and Kishi, et al. Cancer 89:925, 2000).

It is well known to those of skill in the pharmacologic arts that the efficacy of an inhibitor in a patient can rarely be predicted based on a postulated aberrant biochemical pathway, and that many compounds that look promising in the laboratory commonly fail at the bedside. The relationship between AF-NT pathogenesis and a potential therapeutic intervention is even less predictable, since no single pathophysiologic mechanism has been identified. On the contrary, the art teaches that the pathogenesis of AF-NT is multifactorial, and the relative contribution, if any, of particular biochemical derangements has not been defined (see Vesmar et al., Chemotherapy. 49:92, 2003). Moreover, the prior art is contradictory, with different investigators measuring metabolite data in patients afflicted with AF-NT that do not support many of the above biochemical hypotheses. For example, the biopterin deficiency reported in AF-NT and the concommitant deficiency of dopamine and serotonin have not been found by other investigators (see Culvenor, et al., J. Neurochem. 42:1707, 1984; Duch, et al., Mol. Pharmacol. 24:103, 1983; Millot, et al., Pediatr. Res. 37:151, 1995; and Silverstein, et al., Pediatr. Res. 20:285, 1986).

Accordingly, identifying an efficacious therapy for AF-NT is at present an empirical process that can only be undertaken through testing one compound at time, at a defined dose, in a patient with confirmed AF-NT. Some clinical investigators have noted that patients report a resolution of mild, putative AF-NT symptoms using undefined doses of a mixture of CNS-acting drugs (see Bettachi, et al., Arthritis Rheum. 42:S236, 1999). However, since the majority of AF-NT symptoms resolve spontaneously, efficacy of a potential AF-NT therapeutic cannot be demonstrated by only noting symptom resolution or improvement. Further, the art recognizes that efficacy testing must be performed by testing a single compound at a time and at a defined dose, using a validated instrument to quantify patient symptoms (see Tamburini, Ann. Oncol. 12(Suppl. 3):S7). Thus, except for the successful use of the adenosine antagonist aminophylline to treat AF-NT, the prior art provides no teaching of drugs that are efficacious in treating AF-NT in humans (see Bernini et al., Lancet 345:544, 1995 and Peyriere, et al., Med. Pediatr. Oncol. 36:662, 2001).

### SUMMARY OF THE INVENTION

AF-NT is a major short- and long-term complication of MTX therapy. Those of skill in the art will appreciate that AF-NT will also similarly hamper the development and use of other promising antifolates by yielding a similar toxicity in patients as that described for MTX. Methods to treat AF-NT would therefore be highly desirable. Accordingly, there is a need in the art for compositions and methods of using such compositions capable of preventing, reducing, and/or eliminating the toxicity associated with antifolate therapy.

The present invention fulfills this need and further provides other related advantages. It has now been discovered that compounds that block directly or indirectly activities mediated by the NMDA receptor, when administered to a mammal, particularly a human, are effective in treating AF-NT. As used herein, such compounds are referred to as NMDA antagonists. Accordingly, the invention provides methods of treating mammals afflicted with AF-NT, or treating mammals at risk of developing AF-NT, by administering a therapeutically effective dose of an NMDA antagonist. The NMDA antagonist may be administered either before, concurrent with, or sequentially to an antifolate. A partial disclosure of this invention has been provided in Drachtman et al., Pediatr. Hematol. Oncol. 19:319, 2002, incorporated herein by reference for all purposes.

These and other aspects of the present invention will become apparent upon reference to the detailed description and illustrative examples which are intended to exemplify non-limiting embodiments of the invention. All references disclosed herein are hereby incorporated by reference in their entirety as if each was incorporated individually.

### BRIEF DESCRIPTION OF THE DRAWINGS

FIG. 1 is a scatter plot showing the grade of symptom resolution for patients with MTX neurotoxicity who were and were not treated with dextromethorphan (DM). The horizontal lines in each scatter plot represent the mean grade of symptom resolution for that group.

FIG. 2 is a plan view of a package embodying the invention in the form of a dosing card, wherein dextromethorphan and methotrexate are packaged for a four week supply of methotrexate.

FIG. 3 is an enlarged, partial cross-sectional view taken along 2-2 in FIG. 2.

### DETAILED DESCRIPTION OF THE INVENTION

One embodiment of the present invention is a method of treating AF-NT (i.e., antifolate neurotoxicity) in a mammal suffering from or at risk of developing AF-NT, which comprises administering to the mammal a therapeutically effective amount of an NMDA antagonist, or a pharmaceutically acceptable salt thereof. As used herein, the terms “treating AF-NT” and “treating antifolate neurotoxicity” mean the treatment of existing AF-NT in a mammal or the prophylaxis of AF-NT in a mammal at risk of developing AF-NT. Mammals at risk of developing AF-NT are defined as those which are undergoing treatment with an antifolate. Antifolates include, but are not limited to methotrexate, aminopterin, trimetrexate, edatrexate, raltritrexed and lometrexol. As used herein, the terms “NMDA antagonist” and “NMDA receptor antagonist” are used synonymously and mean a compound that reduces NMDA receptor activity by either directly binding to the NMDA receptor, or indirectly affecting its receptor activity. Examples of indirect mechanisms of reducing NMDA receptor activity include a reduction in levels of excitatory NMDA receptor ligands, an allosteric down regulation of the receptor via binding by another protein or receptor, or a down regulation of receptor activity at signaling pathways downstream of the NMDA receptor. An NMDA antagonist may, or may not, also have other CNS and systemic effects in addition to treating AF-NT. The preferred mammal is a human. Thus, the present invention provides a method of treating neoplastic and inflammatory disorders using antifolates while avoiding or reducing the concomitant liability of undesirable AF-NT.

A. Utility and Testing of Compounds of the Invention

Compared to patient populations of the prior art treated with an antifolate but without an NMDA antagonist, the present invention reduces the intensity, frequency and/or duration of one or more AF-NT symptoms in individual patients and patient populations. The intensity, frequency and duration of AF-NT symptoms can be quantified in individual patients using any of a variety of patient self-assessment or physician assessment metrics known to those skilled in the art.

For example, the intensity of neuropsychologic AF-NT symptoms such as memory loss, lethargy, somnolence and headache can be evaluated in patients using validated psychometric and health-related quality of life (HRQOL) testing instruments, such as, for example the Mini-Mental State Examination (MMSE), the Short Test of Mental Status, the EORTC Quality of Life Questionnaire, the FACIT questionnaires and subscales including fatigue and anemia, the Likert Scale, and Borg Scale (Tombaugh, et al., J. Am. Geriatr. Soc. 40:922, 1992; Cummings, JAMA. 269(18):2420, 1993; Crum, et al., JAMA. 269(18):2386, 1993; Folstein, et al., J. Psychiat. Res. 12:189, 1975; Kokmen, et al., Mayo Clin. Proc. 62:281, 1987; Tang-Wai, et al., Arch. Neurol. 60:1777, 2003; Tamburini, Ann. Oncol. 12(Suppl. 3):S7, 2001; Webster et al., Health and Quality of Life Outcomes. 1:79, 2003, www.hqlo.com/content/I/I/79; Grant, et al., Chest. 116:1208, 1999; and www.qolid.org).

These testing instruments may be administered to patients in person, or in some cases remotely via a computer connected to the interne. Each testing instrument has defined measures of differences that have been shown to be reliable and that have been validated. For example, the FACIT instruments have been shown to be responsive to change in both clinical and observational studies. Scores can be interpreted within the context of minimally important differences (MIDs), which are defined as the “smallest difference in score in the domain of interest that patients perceive as important, either beneficial or harmful, and that would lead the clinician to consider a change in the patient's management”. In addition to the ability to evaluate symptom intensity, frequency and duration can also be assessed using these tests by re-administering these tests to patients over time to assess changes in their status as a function of taking an antifolate, either with or without an NMDA inhibitor.

In the case of AF-NT symptoms that result in physical deficits that do not require patient self-assessment, the magnitude, frequency and duration of symptoms can be assessed using standard clinical metrics well known to neurologists and oncologists. For example, the intensity, duration and frequency of neurological symptoms such as seizure, paralysis, nystagmus, ataxia, dysarthria, aphasia cranial nerve palsy, asthenia can be readily determined by clinical evaluation. A variety of validated grading methods are available to assess such symptoms. As used herein, useful strategies for grading such objective AF-NT symptoms include time to initial response, time to resolution of all symptoms, and a grade (1-3) of the rate of symptom resolution for the majority of all AF-NT symptoms. In this grading system, a grade 1 is where symptoms resolve gradually or linearly over an extended period exceeding 96 hours, a grade 2 is where the majority of symptoms resolve in 25 to 96 hours), and a grade 3 is where the majority of symptoms resolve in 24 hours or less: the greater the grade, the greater degree of symptom improvement. Physical deficits that can be objectively assessed generally appear in patients with moderate- to high-dose exposure to antifolate.

Patients with predominantly neuropsychologic AF-NT symptoms commonly arise in the setting of low-dose antifolate exposure as commonly seen in the treatment of rheumatoid arthritis or leukemia in continuation therapy. However, neuropsychologic deficits, particularly IQ deficits, are also a long-term sequelae of treatment with high-dose and even chronic moderate dose antifolate exposure. Not uncommonly, neuropsychologic and physical deficits will exist simultaneously in the same patient. Accordingly, it will be apparent to those skilled in the art that an individual patient and their antifolate dosage history will both need to be evaluated to determine which metric is most suitable to assess their AF-NT symptoms.

As defined herein, an NMDA antagonist is said to be treating AF-NT when it reduces the intensity, frequency or duration of one or more AF-NT symptoms in an individual patient as determined by an appropriate self-assessment or clinical metric. In preferred embodiments, a suitable patient metric reveals a reduction in intensity, frequency or duration of one or more AF-NT symptoms by the invention that is greater than 15%, 25%, 35%, and most preferably greater than 50% of the intensity, frequency or duration of symptoms incurred previously by the patient treated with an antifolate but not with an NMDA antagonist.

An NMDA antagonist is also said to treat AF-NT in a patient population when it reduces the intensity, frequency or duration of one or more AF-NT symptoms in a patient population as determined by a statistically significant change in an appropriate self-assessment and/or clinical metric. In preferred embodiments, a suitable patient metric reveals a reduction in intensity, frequency or duration of AF-NT symptoms by the invention in a patient population that is greater than 15%, 25%, 35%, and most preferably greater than 50% of the frequency or duration of symptoms in a similar patient population treated with an antifolate but not with an NMDA antagonist. The reduction in the intensity, frequency and/or duration of AF-NT symptoms in a patient population as a result of the invention may be expressed as a reduction in the median, mean, or other statistical parameter. In the most preferred embodiments, a double-blinded placebo-controlled clinical trial will reveal a statistically significant change in the intensity, frequency and/or duration of AF-NT upon treatment with an NMDA receptor antagonist that as a P value less than 0.05 using Student's t-test or other suitable statistical analysis.

Accordingly, potential compounds may be tested for their ability to serve as suitable NMDA receptor antagonists by evaluating them using methods described in detail in section B., and also by evaluating whether they treat AF-NT either in a patient or in a patient population as described above. The preferred method of testing the ability of a compound to serve as a suitable NMDA receptor antagonist according to the present invention is by evaluating the compound in a double-blind placebo-controlled clinical trial, and demonstrating a statistically significant change in the intensity, frequency and/or duration of AF-NT (i.e. a P value less than 0.05 using Student's t-test or other suitable statistical analysis). Importantly, only a single compound is given to the test group that is different from the placebo group, so that any efficacy over placebo can be attributable solely to that compound.

As a representative example of how to test whether a compound is suitable as an NMDA antagonist according to the present invention, 200 patients taking weekly methotrexate for rheumatoid arthritis are first evaluated to characterize the intensity, frequency and duration of AF-NT symptoms in relationship to their methotrexate dose. For example, the patients will be evaluated to determine how long after a dose of methotrexate they feel nausea, fatigue, somnolence and experience memory difficulties. These changes will be assessed using the validated FACIT-fatigue instrument, and scores statistically correlated with methotrexate administration by assessing patients just prior to their weekly methotrexate dose and at fixed time points after their dose (e.g. 12, 24, 48, and 72 hours). This will rule out the possibility that symptoms are associated with the underlying disease. With the intensity, frequency and duration of the AF-NT symptoms fully characterized and validated to be associated with methotrexate dosing, these 200 patients are then randomly assigned to two treatment groups.

The first group of 100 patients is given their regular weekly methotrexate dose in the morning together with a single placebo tablet. They are then given a placebo tablet in the evening, and two more placebo tablets the following day, one in the morning and one in the evening. The second group of 100 patients is given their regular weekly methotrexate dose in the morning together with a single 30 mg dextromethorphan tablet. They are then given a 30 mg dextromethorphan tablet in the evening, and two more 30 mg dextromethorphan tablets the following day, one in the morning and one in the evening. Both the patients and the doctors administering the study do not know which tablet is the placebo and which tablet is the 30 mg dextromethorphan. The patient groups are treated in this way for 6 weeks. Each week, all patients in both groups are administered the FACIT-fatigue test the night before their weekly methotrexate dose to establish their pre-methotrexate baseline, and the morning after their methotrexate dose to establish whether there is potential efficacy. The time at which efficacy is assessed will have been established to coincide with AF-NT symptoms.

At six weeks, the placebo and 30 mg dextromethorphan groups are switched in what is known as a cross-over study, such that the group previously receiving placebo now receives 30 mg dextromethorphan, and the group previously receiving 30 mg dextromethorphan now receives placebo. Again, patients and physicians are both blinded. At the end of a second 6 week segment, all the data is collated for each of the two 6-week segments of the study. The scores from the FACIT tests are evaluated both as raw scores and as changes in patient baseline scores for each of the two groups, and in each of the two treatment segments. If the compound is a suitable NMDA antagonist according to the present invention, a Student's t-test of the weekly raw scores and weekly changes in patient baseline scores will show a statistically significant (P<0.05) improvement for patients receiving 30 mg dextromethorphan compared to placebo in both segments of the cross-over study.

B. Compound Embodiments of the Invention Including Preferred Compounds

Direct NMDA Receptor Antagonists

Direct NMDA antagonists are those compounds that bind directly to the NMDA receptor to reduce its activity and include the uncompetitive open channel blocking agents such as memantine or dizocilpine (MK-801). Other uncompetitive NMDA antagonists are, for example, derivatives of dibenzyocycloheptene (Merck; Somerset, N.J.), sigma receptor ligands, for example, dextrorphan, dextromethorphan and morphinan derivatives (Hoffman LaRoche; Nutley, N.J.), such as caramiphen and rimcazole, Ketamine, Tiletamine and other cyclohexanes, remacemide, Phencyclidine (PCP) and derivatives, pyrazine compounds, amantadine, rimantadine and derivatives, CNS 1102 (and related bi-and tri-substituted guanidine), diamines, Conantokan peptide, and Agatoxin-489.

Other direct NMDA antagonists comprise competitive NMDA receptor binding agents, for example, an agent which acts at the agonist binding site, for example, CGS-19755 (CIBA-GEIGY; Summit, N.J.) and other piperidine derivatives, D-2-amino-7-phosphonoheptanoate (AP7), CPP {[3-(2-carboxypiperazin-4-y-propyl-1-phosphonic acid]}, LY 274614, CGP39551, CGP37849, LY233053, LY233536, O-phosphohomoserine, or MDL100-453. Another competitive NMDA receptor binding agent is 2-amino-5-phosphonovalerate (APV). In general, agents that are competitive NMDA receptor binding agents are less preferred because they would be predicted to interfere with the normal physiologic activity of the receptor, and therefore cognition, memory and other important brain functions may be compromised.

Other direct NMDA antagonists include compounds which are active at the glycine site of the NMDA receptor, for example, Kynurenate, 7-chloro-kynurenate, 5,7-chloro-kynurenate, Felbamate, thio-derivatives, and other derivatives (Merck), indole-2-carboxylic acid, DNQX, Quinoxaline or oxidiazole derivatives including CNQX, NBQX, Glycine partial agonist (e.g., P-9939, Hoechst-Roussel; Somerville, N.J.). Also included are NMDA antagonists which are active at the polyamine site of the NMDA receptor: Arcaine and related biguanidines and biogenic polyamines, Ifenprodil, eliprodil, and related drugs, Diethylenetriamine SL 82,0715, or 1,10-diaminodecane and related inverse agonists; and NMDA antagonists which are active at the redox site of the NMDA receptor: oxidized and reduced glutathione, PQQ (pyrroloquinoline quinone). A direct NMDA antagonist should be one which permits the antifolate to remain chemotherapeutically active.

Indirect NMDA Receptor Antagonists

Indirect NMDA receptor antagonists include compounds that generate nitric oxide (NO) or other oxidation states of nitrogen monoxide (NO⁺, NO) such as nitroglycerin and derivatives, sodium nitroprusside, and other NO generating agents, nitric oxide synthase (NOS) inhibitors for example, arginine analogs including N-mono-methyl-L-arginine (NMA), N-amino-L-arginine (NAA), N-nitro-L-arginine (NNA), N-nitro-L-arginine methyl ester, N-iminoethyl-L-ornithine, flavin inhibitors: diphenyliodinium, calmodulin inhibitors, trifluoperizine, calcineurin inhibitors, for example, FK-506 (inhibits calcineurin and thus NOS diphosphorylase). Other non-competitive NMDA antagonists may also be used, for example, 831917189 (Hoechst-Roussel; Somerville, N.J.) and Carvedilol (Smith Kline Beecham; Philadelphia, Pa.).

Other indirect NMDA receptor antagonists include inhibitors of events downstream from activation of the NMDA receptor, for example, agents which inhibit protein kinase C activation by NMDA stimulation may be used: MDL 27,266 (Marion-Merrill Dow; Kansas City, Mo.) and triazole-one derivatives, monosialogangliosides (e.g. GM1 from Fidia Corp., Italy) and other ganglioside derivatives, LIGA20, LIGA4 (may also effect calcium extrusion via calcium ATPase). Also included are agents which inhibit downstream effects from receptor activation to decrease phosphatidylinositol metabolism, such as kappa opioid receptor agonists: U50488 (Upjohn; Kalamazoo, Mich.) and dynorphan, kappa opioid receptor agonist, PD117302 CI-977 or agents which decrease hydrogen peroxide and free radical injury, for example, antioxidants, 21-aminosteroid (lazaroids) such as U74500A, U75412E and U74006F, U74389F, FLE26749, Trolox (water soluble alpha tocopherol), 3-5-dialkoxy-4-hydroxybenzylamines, compounds that generate nitric oxide (NO) or other oxidation states of nitrogen monoxide (NO⁺, NO⁻).

For the purposes of this disclosure, indirect NMDA antagonists will also include agents active at the metabotropic glutamate receptor such as agents that block the receptor, for example, AP3 (2-amino-3-phosphonoprionic acid), or agents that act as agonists of the receptor, for example, (1S,3R)-1-Amino-cyclopentane-1,3-dicarboxylic acid [(1S,3R)-ACPD], commonly referred to as ‘trans’-ACPD. Also included are agents that decrease glutamate release, for example, WEB2086, Y24180, CV6209, adenosine and derivatives such as cyclohexyladenosine, deoxycoformycin, phenytoin, Riluzole, Lamotrgine, Lifarizine, CNS1145, conopeptides: SNX-111, SNX-183, SNX-230, omega-Aga-IVA, toxin from the venom of the funnel web spider, and compounds that generate Nitric Oxide (NO) or other oxidation states of nitrogen monoxide (NO+,NO) as described above. Also included are agents that decrease intracellular calcium following glutamate receptor stimulation, such as agents to decrease intracellular calcium release, for example, dantrolene (sodium dantrium), Ryanodine (or ryanodine+caffeine) or agents that inhibit intracellular calcium-ATPase, for example, Thapsigargin, cyclopiazonic acid, BHQ ([2,5-di-(tert butyl)-1,4-benzohydroquinone; 2,5-di-(tert-butyl)-1,4benzohydroquinone]. Indirect NMDA antagonists will also include calcium channel blockers, such as those that reduce a rise in intracellular calcium. More preferably, the calcium channel blocker is capable of crossing the blood-brain barrier, for example, nimodipine.

In still other embodiments, an indirect NMDA antagonist will include sigma-1 receptor ligands capable of modulating the NMDA receptor. These include the provisionally defined sigma-1 agonists (+)-SKF-10,047, (+)-pentazocine, (+)-3-PPP, imipramine, fluoxetine, fluvoxamine, igmesine, amantadine and memantine and others in Table II. Other sigma-1 receptor ligands that may be suitable as NMDA antagonists according to the present invention include the sigma-1 antagonists dextromethorphan, dextrorphan, dimemorfan, haloperidol, rimcazole, BMS-181100, panamesin, and others in Table III, as well as the as yet undefined sigma-1 ligands eliprodil, ifenprodil, trifluperidol, carbetapentane, caramiphen, dimethoxanate, pipazethate and others in Table IV. Still other sigma-1 receptor ligands are those defined in U.S. Pat. Nos. 6,057,371; 6,087,346; 6,355,659; 6,407,093; 6,417,183; 6,476,019; 6,482,986; and 6,703,383. An indirect NMDA antagonist should be one which permits the antifolate to remain chemotherapeutically active.

Preferred Direct and Indirect NMDA Receptor Antagonists

The preferred compounds of the invention are defined by several key properties. First, they are water soluble and are able to pass readily through the blood brain barrier, facilitating a therapy which is both extremely rapid and unusually potent. Second, the preferred compounds also provide the advantage of a proven record of safe human administration. Finally, where the compounds are direct NMDA receptor antagonists, the inhibitors are low-affinity, open-channel blockers with relatively fast off-rates that substantially preserve non-pathologic NMDA receptor activity and thereby avoid clinically unacceptable side effects. Previously uncharacterized compounds can be evaluated and screened for their suitability as preferred compounds of the invention by screening and evaluating them for being direct and indirect NMDA receptor antagonists, and particularly indirect antagonists that act through the sigma-1 receptor, according to screening procedures and principles set forth in references by Lipton and others (Lipton and Chen, Cell Death and Differentiation. 11:18, 2004; U.S. Pat. Nos. 5,614,560 and 4,806,543; Gamapatju, et al., J. Pharm. Exp. 289:251, 1999; De Haven-Hudkine, et al., Life Science. 53:41, 1993; DeHaven-Hudkine, et al., Eur. J. Pharmacol. 227:371, 1992; de Costa, FEBS 251:53, 1989; Goldman, et al., FEBS Letters. 190:333, 1985). Direct and indirect NMDA receptor antagonists that are found to be suitable according to such criteria are then further tested in humans for efficacy in treating AF-NT according to the guidelines set forth in section A., above.

Accordingly, preferred direct NMDA receptor antagonists are dextromethorphan, dextrorphan, caramiphen, rimcazole, amantadine, rimantadine, memantine, and similar derivatives described in U.S. Pat. Nos. 5,614,560 and 4,806,543. Particularly preferred indirect NMDA receptor antagonists are the sigma-1 ligands dextromethorphan, dextrorphan, dimemorfan, haloperidol, rimcazole, BMS-181100, panamesin, eliprodil, ifenprodil, trifluperidol, carbetapentane, caramiphen, dimethoxanate and pipazethate. Preferred compounds that are both direct and indirect NMDA receptor antagonists often have affinities for the NMDA receptor that are markedly different than their affinity for targets that indirectly antagonize the NMDA receptor. Accordingly, depending on their dose, plasma concentrations can be tailored that result in their action being substantially direct, substantially indirect, or both (see section C. below). As described in greater detail below, pharmaceutical compositions for treating AF-NT in a mammal comprise an amount of an NMDA antagonist (direct or indirect), and more preferably a preferred NMDA antagonist (direct or indirect), or a pharmaceutically acceptable salt thereof.

C. Administration of the Compounds of the Invention

Method of Administration

The present invention encompasses a novel method of treating AF-NT in a mammal, and particularly in a human, which comprises administering an effective amount of an NMDA antagonist. The invention encompasses treating AF-NT by administering an effective amount of an NMDA antagonist either alone or in combination with another pharmaceutical agent, such as an antifolate, another cancer chemotherapeutic, or another AF-NT therapeutic agent or vitamin supplement.

Any suitable route of administration may be employed for providing the patient with an effective dosage of the NMDA antagonist. For example, oral, rectal, parenteral, transdermal, intrathecal, subcutaneous, intramuscular, and the like may be employed as appropriate, using dosage forms that include tablets, coated tablets, troches, dispersions, suspensions, solutions, caplets, capsules, gel capsules, patches, and, the like.

The dosage and dose rate of the NMDA antagonists of this invention will depend on a variety of factors, such as the weight and calculated surface area of the patient, the specific pharmaceutical composition used, the object of the treatment, i.e., treatment or prophylaxis, the judgment of the treating physician, and the response of the individual patient. The dosage of an NMDA antagonist in the acute or chronic management of AF-NT will also vary with the particular NMDA antagonist selected, the severity of the AF-NT symptoms to be treated, the route of administration, as well as the dose and schedule of the antifolate that is causing the AF-NT.

Although not wishing to be limited by theory, it is believed that AF-NT due to typical doses of antifolate (e.g. single doses of 5-25 mg/m² of MTX) can be treated effectively by indirectly antagonizing the NMDA receptor by antagonizing the sigma-1 receptor. This would seem to explain the efficacy of sigma-1 specific doses of dextromethorphan we observed in patients with AF-NT (see Examples). In contrast, neuroprotective effects of dextromethorphan are not seen in hypoxia models until massive doses of dextromethorphan are administered that are sufficient to bind the NMDA receptor directly (Steinberg, et al., Neurol. Res. 15:174, 1993). However, the increases in CNS excitotoxic amino acids during antifolate therapy are relatively minimal compared to the increases associated with hypoxic injury (Quinn et al., J. Clin. Oncol. 15(8):2800, 1997; and Quinn et al., J. Pediatr. Hematol. Oncol. 26:386, 2004). We speculate that the dynamic range of the sigma-1 receptor is relatively small, and therefore capable of effectively down-regulating the NMDA receptor in response to relatively minor antifolate-mediated injury seen with typical doses of antifolate. Larger antifolate doses would be expected to result in proportionally more severe CNS injury, and depending on the dose may require direct antagonism of the NMDA receptor similar to that required in hypoxia models. Those skilled in the art will be familiar on how to upwardly titrate patients from sigma-1 specific doses to doses that directly bind the NMDA receptor in order to satisfactory control AF-NT symptoms.

Thus, in a preferred embodiment of indirect antagonism of the NMDA receptor, either dextromethorphan, dextrorphan, or dimemorfan is used to treat AF-NT in an adult taking 5-25 mg/m² of oral MTX using a daily dose of the NMDA antagonist between 30 and 200 mg, which binds the high-affinity sigma-1 receptor but not the low-affinity NMDA receptor (Steinberg, et al., J. Neurosurg. 84:860, 1996; East and Dye, J. Chromatogr. 338:99, 1985; Barnhart, Toxicol. Appl. Pharmacol. 55:43, 1980). These doses are exceedingly safe, being similar to the doses employed in non-prescription antitussive medications. Generally, daily doses of dextromethorphan, dextrorphan, or dimemorfan between 0.15 mg/kg and 5 mg/kg will produce a substantially indirect antagonism of the NMDA receptor via binding to the high-affinity sigma-1 receptor.

In a preferred embodiment of direct and indirect antagonism of the NMDA receptor, either dextromethorphan or dextrorphan is used to treat AF-NT in an adult using a daily dose between 10-20 mg/kg to affect plasma concentrations between 1 and 5 μM (Steinberg et al., J. Neurosurg. 84:860, 1996). These plasma concentrations result in substantial binding to both the NMDA (dextromethorphan Kᵢ=7 μM and dextrorphan Kᵢ=1 μM) and sigma-1 receptors (dextromethorphan Kᵢ=0.21 μM and dextrorphan Kᵢ=0.14 μM). Although, these doses have been shown to produce a variety of PCP-like symptoms, they are relatively safe as these side effects have been shown to be completely reversible (Albers et al., Clin. Neuropharmacol. 15(6):509, 1992; Albers et al. Stroke. 26:254, 1995; and Steinberg et al., J. Neurosurg. 84:860, 1996). Generally, daily doses of dextromethorphan, dextrorphan, or dimemorfan greater than about 10 mg/kg will produce a substantially direct and indirect antagonism of the NMDA receptor via binding to both the NMDA receptor directly as well as the high-affinity sigma-1 receptor.

In yet another preferred embodiment of direct and indirect antagonism of the NMDA receptor, memantine is used to treat AF-NT in an adult using a daily doses between 5 and 30 mg to affect plasma concentrations between 0.025 and 0.529 μM (Kornhuber and Quack, Neurosci. Lett. 195:137, 1995). With a mean CSF/serum ratio of 0.52, these plasma concentrations result in substantial binding to both the NMDA and sigma-1 receptors, since both have essentially equivalent affinity for memantine (Kornhuber and Quack, Neurosci. Lett. 195:137, 1995; Kornhuber, et al., Neurosci. Lett. 163(2):129, 1993; and Peeters, et al., Eur. J. Neurosci. 19:2212, 2004). These doses have been shown to have a high degree of safety, being FDA approved for patients with moderate to severe Alzheimer disease (Tariot et al., JAMA. 291(3):317, 2004). Generally, daily doses of memantine and amantadine greater than about 10 mg per day will produce a substantially direct and indirect antagonism of the NMDA receptor via binding to both the NMDA receptor directly as well as the sigma-1 receptor.

In general, in the case where an oral administration of an NMDA antagonist is employed, a suitable dosage range for use in treating AF-NT is, for example, from about 0.1-20 mg/kg daily, 1-5 mg/kg daily, and more preferably 0.5-2 mg/kg daily. In a specific preferred embodiment, the NMDA antagonist is dextromethorphan administered to a patient in a single dose of 1-2 mg/kg, in multiple doses of 1-2 mg/kg daily, or 1 mg/kg three times a day for a single day or daily in order to treat MTX neurotoxicity arising from MTX doses that range from 5 mg to 12 grams. Based on the known pharmacology, pharmacokinetics, and toxicology of a particular NMDA antagonist, a skilled practitioner can select an appropriate NMDA antagonist to treat AF-NT and established a safe and effective dose and dose schedule.

Patients may be upward titrated from below to within these dose ranges to a satisfactory control of symptoms. It is recommended that patients over age 65, and those with impaired renal or hepatic function, initially receive low doses. In some cases, it may be necessary to use dosages outside these ranges. For example, in some embodiments, dosages up to 50 mg/kg may need to be administered.

Once improvement in the patient's condition has occurred, a maintenance dose of an NMDA antagonist may be employed for the duration of antifolate exposure. Subsequently, the dosage or the frequency of administration, or both, may be reduced, as a function of the AF-NT symptoms, to a level at which the improved condition is retained. When the AF-NT symptoms have been alleviated to the desired level, the practitioner may elect to cease treatment. Patients may, however, require intermittent treatment upon any recurrence of AF-NT symptoms, or require prophylactic scheduled treatments as required.

Various embodiments of the invention thus encompass the long-term use of an NMDA antagonist during the long-term treatment of a neoplastic or inflammatory disorder with an antifolate. These embodiments rely on the use of weekly or daily, or other regular small interval sub-toxic dosage of an NMDA antagonist over a long period of time, ranging up to 36 months, or even longer.

In one embodiment, prophylaxis of AF-NT is accomplished by administering an NMDA antagonist in a dosage that has a duration shorter than the duration of efficacy resulting from a particular anti folate dosage. Thus, it has been unexpectedly found that AF-NT prophylaxis may be accomplished during repetitive dosages of antifolate by administering an NMDA antagonist in a series of doses whose duration is shorter than the duration of efficacy of the antifolate dosages. For example, AF-NT prophylaxis may be accomplished during weekly low-dose methotrexate therapy by a dosage of NMDA antagonist whose schedule is interrupted by periods of from 4 to 6 days where no NMDA antagonist is administered. This was surprising given that the efficacy of weekly methotrexate continues uninterrupted from week to week.

In a particularly preferred embodiment, dextromethorphan is used as prophylaxis of AF-NT in patients taking chronic weekly low-dose methotrexate (5-100 mg/m² of oral MTX weekly) for conditions such as rheumatoid arthritis, juvenile rheumatoid arthritis, or leukemia. Such patients take their weekly methotrexate as a single dose or as a plurality of divided doses over a period of one to two days. To provide AF-NT prophylaxis in these patients, a plurality of 15 to 60 mg doses of dextromethorphan are given to the patient during the time period ranging from about 5 hours prior to their first weekly dose of methotrexate to about 72 hours after their last weekly dose of methotrexate. This prophylaxis is then repeated weekly as long as the patient continues to be treated with MTX.

In a specific embodiment of AF-NT prophylaxis, a patient with rheumatoid arthritis takes a weekly morning dose of 5-25 mg/m² oral MTX together with a 30 mg dose of dextromethorphan, and optionally 1 mg of folic acid. The patient then takes a 30 mg dose of dextromethorphan that evening, the next morning, and the next evening for a total of four 30 mg dextromethorphan doses per week.

Pharmaceutical Compositions

The pharmaceutical compositions of the present invention comprise an NMDA antagonist as the active ingredient, or a pharmaceutically acceptable salt thereof, and may also contain a pharmaceutically acceptable carrier, and optionally, other pharmaceutical agents and vitamin supplements.

“Pharmaceutically acceptable salts” refers to salts prepared from pharmaceutically acceptable non-toxic acids or bases including inorganic acids and bases and organic acids and bases. Since the NMDA antagonists of the present invention may be either basic or acidic, salts may be prepared from pharmaceutically acceptable non-toxic acids or bases including inorganic and organic acids or inorganic and organic bases. Such salts may contain any of the following anions: acetate, benzensulfonate, benzoate, camphorsulfonate, citrate, fumarate, gluconate, hydrobromide, hydrochloride, lactate, maleate, mandelate, mucate, nitrate, pamoate, phosphate, succinate, sulfate, tartrate and the like. Such salts may also contain the following cations: aluminum, calcium, lithium, magnesium, potassium, sodium, zinc, benzathine, chloroprocaine, choline, diethanolamine, ethylenediamine, meglumine, and procaine.

Pharmaceutical carriers can be combined in intimate admixture with the NMDA antagonist as the active ingredient according to conventional pharmaceutical compounding techniques. The carrier may take a wide variety of forms depending on the form of the preparation desired for administration, e.g., oral, parenteral, or intrathecal (including intravenous injections or infusions). In preparing the compositions for oral dosage form any of the usual pharmaceutical media may be employed. Usual pharmaceutical media includes, for example, water, glycols, oils, alcohols, flavoring agents, preservatives, coloring agents, and the like in the case of oral liquid preparations (such as for example, suspensions, solutions, and elixirs); aerosols; or carriers such as starches, sugars, microcrystalline cellulose, diluents, granulating agents, lubricants, binders, disintegrating agents and the like, in the case of oral solid preparations (such as for example, powders, capsules, and tablets) with the oral solid preparations generally being preferred over the oral liquid preparations. The most preferred oral solid preparation is tablets. For pediatric patients, it will be appreciated to those skilled in the art that pleasant tasting oral liquid preparations are preferred.

Other pharmaceutical agents and vitamin supplements suitable from inclusion in the pharmaceutical compositions of the present invention include but are not limited to methotrexate, aminopterin, trimetrexate, edatrexate, raltritrexed, lometrexol, leucovorin, S-adenosyl-methionine, betaine, vitamin B₆, vitamin B₁₂, aminophylline, tetrahydrobiopterin, L-dopa, carbidopa, 5-hydroxytryptophan, one or more additional NMDA antagonists of the invention, doxorubicin, cisplatin, ifosfamide, paclitaxel, 5-fluoruracil, etoposide, dianydrogalacitol, tamoxifen, piperazinedione, mitoxantrone, diaziquone, aminothiadiazole, tenoposide, vincristine, echinomycin, 6-mercatopurine, dexamethasone, cyclophosphamide, cytaribine, L-asparaginase, non-steroidal anti-inflammatory compounds, soluble TNF receptors, antibodies, and humanized antibodies. In a preferred embodiment, the NMDA antagonist is used in combination with an agent that slows its metabolism and therefore prolongs its serum half-life. Suitable strategies and agents for slowing the metabolism of an NMDA antagonist are provided in U.S. Pat. No. 5,166,207, incorporated herein by reference.

The pharmaceutical compositions include those suitable for oral, rectal, intrathecal and parenteral (including subcutaneous, intramuscular, and intravenous) administration, although the most suitable route in any given case will depend on the nature of the NMDA antagonist employed, and the nature and severity of the AF-NT being treated. For example, NMDA antagonists that cannot cross the blood/brain barrier may be administered locally (i.e. intrathecally). Agents capable of crossing the blood/brain barrier, for example, nimodipine and dextromethorphan, can be administered systemically, for example, orally or intravenously. The most preferred routes of the present invention are the oral and intrathecal routes. The compositions may be conveniently presented in unit dosage form, and prepared by any of the methods well known in the art of pharmacy.

Pharmaceutical compositions of the present invention suitable for oral administration may be presented as discrete units such as capsules, gel capsules, cachets, or tablets, or aerosols sprays, each containing a predetermined amount of the active ingredient, as a powder or granules, or as a solution or a suspension in an aqueous liquid, a non-aqueous liquid, an oil-in-water emulsion, or a water-in oil liquid emulsion. Such compositions may be prepared by any of the methods of pharmacy, but all methods include the step of bringing into association the active ingredient with the carrier that constitutes one or more necessary ingredients. In general, the compositions are prepared by uniformly and intimately admixing the active ingredient with liquid carriers or finely divided solid carriers or both, and then, if necessary, shaping the product into the desired presentation or dosage form.

D. Dosage Forms and Preferred Multiple Dosage Forms

Due to their ease of administration, tablets, capsules and gel capsules represent the most advantageous oral dosage unit form, in which case solid pharmaceutical carriers are employed. If desired, tablets may be coated by standard aqueous or nonaqueous techniques. The parenteral dosage form can consist of a sterile solution of the active ingredient, either in its free or salt form, in physiological buffer or sterile water. In addition, parenteral solutions can contain preservatives such as benzalkonium chloride, methyl- or propyl-paraben and chlorobutanol. Suitable pharmaceutical carriers are described in Remington's Pharmaceutical Sciences, a standard reference text in this field.

For example, a tablet may be prepared by compression or molding, optionally, with one or more accessory ingredients. Compressed tablets may be prepared by compressing in a suitable machine the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Desirably, each tablet, cachet, or capsule contains from about 10 mg to about 100 mg of the NMDA antagonist. Most preferably, the tablet, cachet or capsule dosage forms contain one of four dosages consisting of either about 15 mg, 30 mg, 60 mg, or about 100 mg of dextromethorphan.

In addition to the common dosage forms set out above, the NMDA antagonists of the present invention may also be administered by controlled release means and/or delivery devices such as those described in U.S. Pat. Nos. 3,845,770; 3,916,899; 3,536,809; 3,598,123; 3,630,200; 4,008,719; 4,687,660 and 4,769,207, the disclosures of which are hereby incorporated by reference.

A combination dosage form as used herein includes a single dosage form containing at least one NMDA antagonist of this invention and at least one other pharmaceutical agent in intimate admixture with one another. A combination dosage form also includes a multiple dosage form, wherein the NMDA antagonist and the pharmaceutical agent are administered separately, but concurrently, or a multiple dosage form wherein the two components are administered separately, but sequentially (see preferred multiple dosage forms below). In preferred embodiments the pharmaceutical agent at least comprises an antifolate, although it may comprise any of the pharmaceutical agents and vitamin supplements described in section C., above. In sequential administration, the NMDA antagonist may be given to the patient one or more times during the time period ranging from about 5 hours prior to about 200 hours after administration of the pharmaceutical agent.

In embodiments where the pharmaceutical agent at least comprises an antifolate, the combination dosage form directs the NMDA antagonist to be administered to the patient in a plurality of doses during a period that is shorter than the period of antifolate efficacy. In a preferred embodiment where the antifolate is administered in one or more weekly doses, the combination dosage form directs the NMDA antagonist to be administered to the patient in a plurality of doses during a period ranging from about 5 hours prior to the first weekly antifolate dose to 72 hours following administration of the last weekly antifolate dose. The dosage form further directs the patient to continue with this protocol weekly as long as the antifolate is being taken. This combination dosage form has been unexpectedly found to prophylax AF-NT symptoms during weekly antifolate therapy by a dosage of NMDA antagonist whose schedule is interrupted by periods of from 4 to 6 days where no NMDA antagonist is administered even though the efficacy of the antifolate continues uninterrupted from week to week.

One preferred multiple dosage form of the present invention contemplates a package or dosing card for sequential weekly oral administration of an antifolate together with an NMDA antagonist, and optionally other supplements, comprising a carrier sheet provided with substantially duplicate groups of compartments except for certain indicia, wherein each group of compartments contains a week supply of the above antifolate, NMDA antagonist, and other optional supplements. Each grouping of compartments is arranged in substantially parallel rows of from one to a plurality of substantially evenly spaced compartments, wherein each compartment in a particular row lies in a particular column of from one to a plurality of substantially evenly spaced compartments. Thus, some compartments from different rows are aligned with one another to form a plurality of compartments in a column, while other columns may comprise only a single compartment from a row.

The one or more compartments in a row each contain the same active tablet, wherein a first row contains an NMDA antagonist, a second row contains an antifolate, and an optional third or more rows each contains a supplement such as folic acid or other vitamin. The rows and columns are provided with indicia that identify the active tablet in each row and the day of the week each tablet is to be taken by the patient, respectively. Tablets in compartments that are aligned in a column are intended to be taken simultaneously as indicated by the column indicia.

Preferably the carrier sheet is about the size of a credit card, so as to provide the patient with a convenient means of carrying the dosing card. Optionally, the dosing card is provided with lines of severability to permit individual groupings to be separated from one another and carried as a single weekly supply. In a further optional embodiment, the dosing card is part of a plurality of dosing cards provided as a roll of dosing cards, wherein a dosing card at the end of the role may be detached through a line of severability.

With both these optional embodiments, dosing cards with lines of severability on a roll allows pharmacists to fill a physician's prescription for a plurality of separate weekly packages, a monthly dosing card, or combination thereof using a single packaging unit that the pharmacist simply tears along different lines of severability depending on the physician's prescription. The pharmacy therefore needs to stock only one package type to fill either prescription, thereby reducing its inventory. Further, the drug manufacturer needs only produce one type of package, thereby lowering costs, minimizing inventory, and decreasing the investment in inventory for manufacturers, the wholesalers, and chains and individual pharmacies.

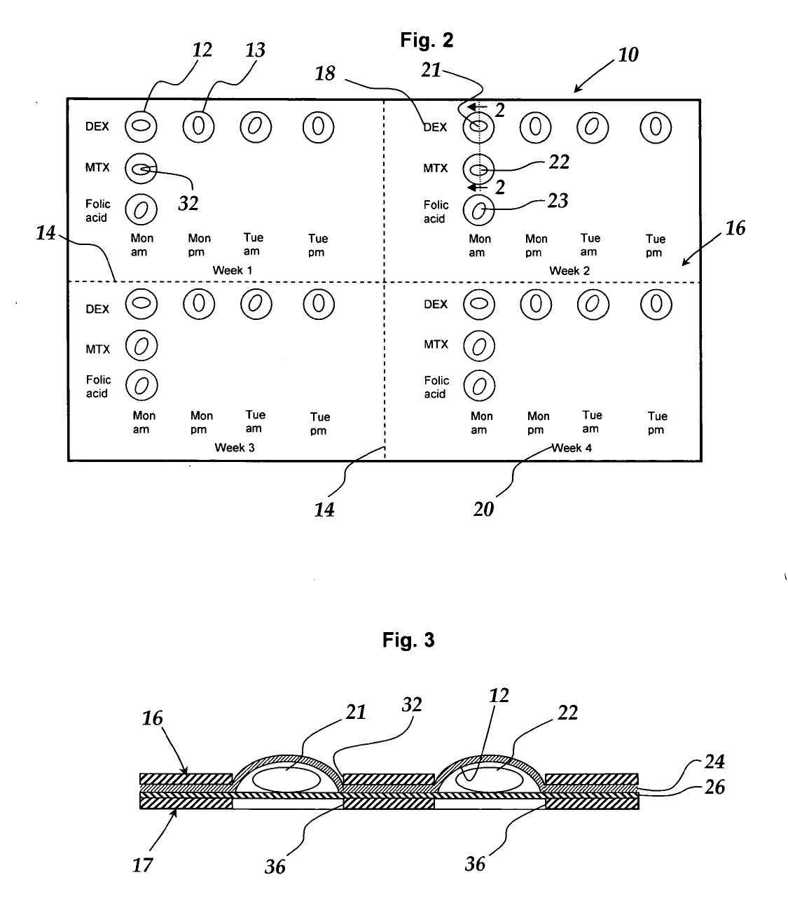

A preferred embodiment of the present invention is illustrated in FIG. 2. Package 10 is provided with plural compartments 12 arranged in rows and columns. For convenience, and to provide a medication regimen for 4 weeks, compartments 12 are arranged in four substantially duplicate groups of parallel rows and generally parallel columns. Each compartment 12 contains a daily dosage of therapeutic medication or optional supplement to be administered, wherein all compartments in a particular row within a group each contain the same active tablet.

An apertured panel 16 is provided with medication and supplement indicia 18 and daily and weekly indicia 20. These indicia tell the patient what medication they are taking, and when in the week they are taking the medication. As can be seen in FIG. 2, each group contains six compartments 12 arranged to form three rows each containing a week's supply of tablets such as tablets 21, 22, and 23. Tablet 21 contains an NMDA antagonist (‘DEX’ or dextromethorphan in this embodiment) and tablet 22 contains an antifolate (‘MTX’ or methotrexate in this embodiment). Optional tablet 23 contains a supplement (‘folic acid’ in this embodiment). If desired, tablets in a row can have coloring different from tablets in other rows to distinguish them. The tablets in the same column are to be taken together according to the daily and weekly indices 20 (DEX, MTX and folic acid on Mon, am for each week in this embodiment).

This arrangement of compartments provides a relatively reliable system with a built-in feedback mechanism in that the patient can readily determine if they have taken the proper tablets on proper days for the proper week by comparing the day of the week with the appropriate indicia on the package. The patient can easily recognize that a day's dosage has been missed, or if more than one dose has been inadvertently taken on a particular day.

Package 10 is provided with lines of severability 14 that traverses package 10 in the horizontal and longitudinal directions. Lines of severability 14 are situated between each of the groupings of package 10. In a more preferred embodiment, lines of severability 14 is a line of weakening, thereby facilitating severing the weekly grouping of tablets along the line; however, the line of severability can also be a crease, a fold line, or the like. The line of weakening can be provided by a line of partial cuts or by a line of perforation.

The construction of package 10 can be best seen by reference to FIG. 3. Carrier sheet 24, preferably made of a transparent material, defines each compartment 12. A tablet, such as tablet 22, is received in each compartment 12 and is retained therein by cover 26 that seals compartment 12 from ambient surroundings. If the carrier sheet material is of a sufficiently heavy gauge, no further support is necessary. If a relatively lighter gauge material is desired, one or two apertured panels of a relatively stiff material such as cardboard as illustrated by apertured panels 16 and 17 are provided to sandwich carrier sheet 24 and its associated cover or covers 26 therebetween so as to enhance the overall rigidity of package 10.

Panel 16 defines plural apertures 32 through which extend flexible protrusions or indentations in carrier sheet 24 that define compartments 12. Corresponding apertures 36 are provided in panel 17 and define openings through which the tablets such as tablets 21 and 22 in compartments 12 can be dispensed as cover 26 is ruptured, by flexing or deforming indentations 11 for example. Apertures 32 and apertures 36 are in substantially registry with respect to one another.

Weekly, daily and time (am vs. pm, for example) indicia such as 20 can be provided at each column and at each weekly grouping. Drug and supplement indicia such as 18 are provided adjacent to each row. Antifolate formulations are often designed to be taken on a predetermined day or predetermined days of the week, and then weekly thereafter on the same predetermined day or days. For example, the treatment of rheumatoid arthritis, juvenile rheumatoid arthritis and leukemia (in continuation therapy) all typically employ a dose of methotrexate taken weekly on one or two predetermined days per week. Alternative indicia 20 different from those shown in FIG. 2 are therefore also possible and will be determined by the particular antifolate dosage schedule of a particular disease.

Numerous ways to facilitate indicating the day of each tablet are known in the art. For example, U.S. Pat. No. 3,397,671 provides a holder having two rows with 16 daily indicia per row into which a 10 tablet carrier is inserted. The holder is placed over the carrier so that the daily indicia labeling the tablets are appropriate for a particular woman taking the tablets. As another example, U.S. Pat. No. 3,494,322 discloses a package having four rows of seven compartments. A separate strip with 13 daily indicia can be pulled through a support card that has holes cut out above each column of labels through which the daily indicia can be viewed. The patient aligns the strip as required to expose only the needed indicia. Other similar embodiments can be used. For example, an adhesive strip with daily and weekly indicia can be applied by the patient or an easily labeled surface can be placed atop each column for the patient to mark.

Thus, package 10 and other such packaging embodiments will have significant utility in providing patients with a convenient mechanism to treat their AF-NT within a variety of antifolate treatment scenarios.

The invention is further defined by reference to the following examples describing in detail, the methods and the compositions of the present invention. It will be apparent to those skilled in the art, that many modifications, both to materials, and methods, may be practiced without departing from the purpose and interest of this invention.

### EXAMPLES

### Example 1

### Patient with AF-NT Treated with an NMDA Antagonist According to the Invention (Table V)

A 32 year old male with non-Hodgkin's lymphoma was treated with 12 mg intrathecal MTX and 2 days later developed headache, dysarthria, nausea, weakness and asthenia. The patient was treated once with 1 mg/kg dextromethorphan. His symptoms began to improve after 3 hours, and the duration of his symptoms was 24 hours. He had a grade 3 symptom resolution.

### Example 2

### Patient with AF-NT Treated with an NMDA Antagonist According to the Invention (Table V)

A 15 year old male with acute lymphoblastic leukemia was treated with 12 mg intrathecal MTX and 100 mg/m² intravenous MTX. He developed a left hemiparesis 7 days after treatment and was treated once with 2 mg/kg dextromethorphan. His symptoms began to improve after a half-hour, and the duration of his symptoms was 6 hours. He had a grade 3 symptom resolution.

### Example 3

### Patient with AF-NT Treated with an NMDA Antagonist According to the Invention (Table V)

A 19 year old male with acute lymphoblastic leukemia was treated with 7.5 mg intrathecal MTX (via an Ommaya device) and 1 g/m² intravenous MTX. He developed a right cranial nerve VII palsy, right hemiparesis, and dysarthria 12 days after treatment and was treated with 1 mg/kg dextromethorphan three times on a single day. His symptoms began to improve after 3 hours, and the duration of his symptoms was 10 days. He had a grade 1 symptom resolution.

### Example 4

### Patient with AF-NT Treated with an NMDA Antagonist According to the Invention (Table V)

A 13 year old male with osteosarcoma was treated with 12 g/m² intravenous MTX. He developed a right cranial nerve VII palsy, left hemiparesis, dysarthria, and an impaired gag reflex 7 days after treatment with MTX. He was treated with 1 mg/kg dextromethorphan three times on a single day. His symptoms began to improve after 45 minutes, and the duration of his symptoms was 3 days. He had a grade 2 symptom resolution.

### Example 5

### Patient with AF-NT Treated with an NMDA Antagonist According to the Invention (Table V)

A 16 year old male with osteosarcoma was treated with 12 g/m² intravenous MTX. He developed a right cranial nerve VII palsy and dysarthria 7 days after treatment with MTX. He was treated once with 1 mg/kg dextromethorphan. The duration of his symptoms was 30 minutes. He had a grade 3 symptom resolution.

### Example 6

### Patient with AF-NT Treated with an NMDA Antagonist According to the Invention (Table V)

An 18 year old Latina girl with high risk acute lymphoblastic leukemia (CNS negative) did well through induction, consolidation and interim maintenance during which she received chemotherapy including intrathecal and oral MTX. Six months into her therapy, during delayed intensification, she received intrathecal MTX 12 mg and 4 days later presented with disorientation and rambling slurred speech. She was started on dextromethorphan (1 mg/kg po BID). Although she had some improvement in her symptoms in 24 hr, she had persistent confusion for 8 days. No further intrathecal MTX given, and the patient had no recurrence of symptoms. She had a grade 2 symptom resolution.

### Example 7

### Patient with AF-NT Treated with an NMDA Antagonist According to the Invention (Table V)

A 6 year old Latina girl with standard risk acute lymphoblastic leukemia (CNS negative) received chemotherapy including intrathecal and oral MTX during induction, consolidation and interim maintenance without difficulties. Six months into her therapy she received a dose of intrathecal MTX 12 mg. Five days later she presented with paresis of her R upper extremity and right face. She was treated with dextromethorphan (1 mg/kg po BID×3 doses). Her symptoms resolved completely within 4 hours and she never returned. She had a grade 3 symptom resolution.

### Example 8

### Patient with AF-NT Treated with an NMDA Antagonist According to the Invention (Table V)

A 9 year old Caucasian girl with standard risk ALL (CNS negative). She was treated without difficulty during the first two months of her therapy, with chemotherapy including intrathecal MTX. About 8 weeks into her therapy she received intrathecal MTX 12 mg and 4 days later she presented with slurred speech, drooling, headache and left arm weakness as well as ataxia. She was started on dextromethorphan (1 mg/kg po BID). Her symptoms waxed and waned within hours but did not completely resolve until at least 48 hours. These problems did not recur. She had a grade 2 symptom resolution.

### Example 9

### Patient with AF-NT Treated with an NMDA Antagonist According to the Invention (Table V)

A 14 year old Caucasian boy with high risk ALL (CNS negative) was treated without difficulty during the first 5 months of his therapy. He was treated with chemotherapy including intrathecal and intravenous MTX without complications. About 5½ months into his therapy he received intravenous and intrathecal MTX and 12 days later presented with a tonic clonic seizure. He was started on dextromethorphan (1 mg/kg po TID×24 hours) and within hours he was completely back to his baseline. He had a grade 3 symptom resolution.

### Example 10

### Patient with AF-NT Treated with an NMDA Antagonist According to the Invention (Table V)

An 8 year old Latina girl with high risk ALL (CNS negative) was started on induction chemotherapy. She began to have headaches within a few days of her initial chemotherapy as well as intermittent hypertension of unclear etiology. She started on her chemotherapy and received her first dose of intrathecal MTX 12 mg. Two days later she became disoriented, with roving hemiparesis that was more impressive on the right. She was started on dextromethorphan (1 mg/kg po BID for 24 hours). Her neurologic problems improved within the first 36 hours, but did not resolve completely for 5 days. She had a grade 2 symptom resolution.

### Example 11

### Patient with AF-NT Treated with an NMDA Antagonist According to the Invention (Table V)

A 15 year old Latina girl with high risk ALL (CNS negative) did well during her initial chemotherapy, which included intrathecal and oral MTX. Two months into her therapy she received intrathecal MTX 12 mg and four days later presented with right-sided weakness. Dextromethorphan was started (1 mg/kg po BID) and within the first day she had dramatic improvement. She continued to have some subtle weakness ongoing, but was back to her baseline at 8 days. She had a grade 3 symptom resolution.

### Example 12

### Patient with AF-NT Treated with an NMDA Antagonist According to the Invention (Table V)

A 5 year old Caucasian boy with high risk ALL (CNS negative) tolerated his initial chemotherapy including intrathecal MTX. About five months into his therapy he received intravenous and intrathecal MTX. Eight days later he had a seizure at home. On presentation he was post-ictal and had a dysconjugate gaze for hours afterward. He was started on dextromethorphan (1 mg/kg po TID). His symptoms resolved the first day and the medication was discontinued after 24 hours. He had a grade 3 symptom resolution.

### Example 13

### Patient with AF-NT Treated with an NMDA Antagonist According to the Invention (Table V)

A 13 year old Latino boy with high risk ALL (CNS negative) tolerated his first 2 months of chemotherapy, which included intrathecal MTX. About 8 weeks into his therapy he received intrathecal MTX 12 mg and 3 days later presented in a disoriented state (may have been post-ictal, but no seizure had been witnessed or reported). He was started on dextromethorphan (1 mg/kg po BID) and had a normal neurologic exam the next morning. He had a grade 3 symptom resolution.

### Example 14

### Patient with AF-NT Treated with an NMDA Antagonist According to the Invention (Table V)

A 14 year old Latina girl with high risk ALL (CNS negative) tolerated chemotherapy including intrathecal MTX for two months. During her third month of therapy she received intrathecal MTX 12 mg and 7 days later presented with mental status change (difficulty with word-finding), decreased level of consciousness, slurred speech and a left facial droop. Over the next few hours she progressed to being unable to speak and she developed right-sided weakness. Dextromethorphan (1 mg po BID) was started by nasogastric tube since she was having difficulty with her gag reflex as well. She showed very slow improvement. By 9 days she was able to speak, and she continued to have a mild right hemiparesis. These residual symptoms took several months to resolve. She had a grade 1 symptom resolution.

### Example 15

### Patient with AF-NT Treated with an NMDA Antagonist According to the Invention (Table V)

A 13 year old Caucasian girl with high risk ALL (CNS negative) tolerated her initial chemotherapy which included intrathecal MTX for about 10 weeks. She then received intrathecal MTX 12 mg and 9 days later was admitted with altered mental status, including confusion and combative behavior with slurred speech. She has a neurological work-up which was negative. Over several days these symptoms resolved, and were attributed to an adverse drug reaction to a non-chemotherapeutic medication. Approximately one month after her first neurologic event, she received intrathecal MTX 12 mg. Nine days later she was admitted with difficulty swallowing and talking. She developed a right hemiparesis. She became more agitated requiring Haldol and benzodiazepines to prevent her from harming herself. She was non-verbal for several weeks and had non-purposeful movements of her extremities. She developed spasticity. Nine days after her symptoms began she was started on dextromethorphan (1.5 mg/kg po BID). She had more periods of alertness and focused concentration. After 6 days the dextromethorphan was discontinued. Three weeks into the hospitalization she had increasing but still slow abnormal speech, she was able to stand and walk but only with assistance. She had a grade 1 symptom resolution.

### Example 16

### Patient with AF-NT Treated with an NMDA Antagonist According to the Invention (Table V)

A 6 year old Caucasian/Japanese boy with high-risk ALL (CNS negative) had an initial white cell count of 100,000 which rapidly increased to 200,000 within 12 hours of starting his initial chemotherapy. He developed a headache with hypertension and had a seizure about 72 hours after he began systemic chemotherapy and 48 hours after he received intrathecal cytosine arabinoside. His neurologic exam became normal within 24 hours. One week later he continued on his systemic chemotherapy and he received intrathecal MTX 12 mg and two days later he developed dysmetria, ataxia and right upper extremity weakness. He was started on dextromethorphan (1 mg/kg po BID) for 5 days. His symptoms improved within 24-48 hours but did not completely resolve for about 5 days. This is the only patient among the Examples who has ongoing neurologic concerns, though it is felt that he actually had a stroke with his first neurologic event (he had not yet received MTX). He had a grade 1 symptom resolution.

### Examples 17-30

### Patients with AF-NT not Treated with an NMDA Antagonist According to the Prior Art (Table V)

The same patient as in Example 1, but months earlier, was treated with 12 mg intrathecal MTX, but was not treated with dextromethorphan (Example 17). Approximately 2 days later he developed headache, dysarthria, nausea, weakness and asthenia. The time to an initial improvement was 48 hours, and the duration of his symptoms was 336 hours. He was judged to have a grade 1 symptom resolution. For Examples 18-30, the clinical histories of patients with AF-NT who were not treated with an NMDA antagonist were obtained from a variety of published sources (see Yim et al., Cancer 67:2058, 1991; Packer et al., Medical and Pediatric Oncology 11:159, 1983; Jaffe et al., Cancer 56:1356, 1985; and Gay et al., J. Child Neurol. 4:207, 1989, includes Ara-C as part of intrathecal therapy).

### Example 31

### Comparison of Patients with AF-NT Treated and Not Treated with an NMDA Antagonist

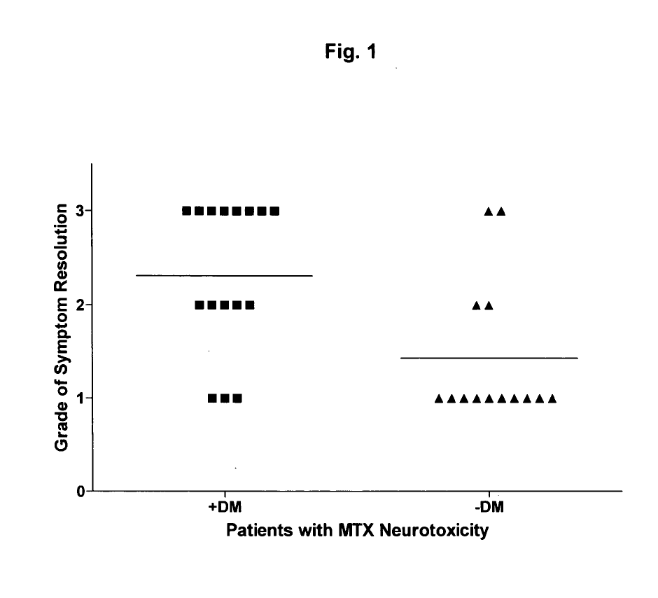

The grade of symptom resolution was plotted for patients with AF-NT treated (Examples 1-16) and not treated (Examples 17-30) with the NMDA antagonist dextromethorphan (FIG. 1). The mean grade of symptom resolution for untreated patients was 1.4±0.2 (mean±SEM), while the mean grade of symptom resolution for treated patients was 2.3±0.2 (mean±SEM). Using an unpaired Students t test, the means were found to be significantly different (P value of 0.0042).

### Example 32

### Pharmaceutical Compositions

This example illustrates the preparation of representative pharmaceutical compositions containing NMDA antagonists, or a pharmaceutically acceptable salt thereof:

Tablets: Combine 1600 grams of dextromethorphan, 3219 grams microcrystalline cellulose, 2946 grams lactose monohydrate, 15.9 grams colloidal silicon dioxide, 188 grams sodium croscarmellose, and 31.3 grams magnesium stearate to provide a total weight 8000 grams. Compress into approximately 16,000 tablets using a tableting machine, wherein each tablet weighs approximately 500 mg and contains 100 mg of dextromethorphan.

Gelatin capsules: Prepare by mixing 50 grams of dextromethorphan with 25 grams magnesium stearate, and 225 grams of lactose. Dispense 300 mg of this mixture into hard-shell gelatin capsules to provide each capsule with 50 mg of dextromethorphan.

Suspension: An aqueous suspension is prepared for oral administration so that each 5 ml contains 20 mg of nimodipine by mixing 400 mg of nimodipine with 20 grams of sodium carboxymethyl cellulose, 0.5 grams of sodium benzoate, 100 grams of sorbitol solution U.S.P., and 2.5 ml of vanillin.

Intrathecal formulation: An intrathecal formulation is prepared by mixing 1 grams of sterile memantine with a 100 ml of sterile Elliot's B solution (buffered intrathecal electrolyte/dextrose solution). Per 100 ml, Elliot's B solution is 730 mg sodium chloride, 190 mg sodium bicarbonate, 80 mg dextrose, 30 magnesium sulfate-7H₂0, 30 mg potassium chloride, 20 mg calcium chloride-2 H₂0, sodium phosphate dibasic-7H₂0). Package under sterile conditions in 2-ml vials each containing 1 ml, or a total of 10 mg/vial.

The present invention is not to be limited in scope by the specific embodiments disclosed in the examples which are intended as illustrations of a number of aspects of the invention and any embodiments which are functionally equivalent are within the scope of this invention. Indeed, various modifications of the invention in addition to those shown and described herein will become apparent to those skilled in the art and are intended to fall within appended claims.

A number of references have been cited, the entire disclosures of which are incorporated herein by reference.

TABLE II
Sigma-I Agonists.

| | | | Used in | |
|---|---|---|---|---|
| | sigma-1 | | animals (a) or | |
| Compounds | affinity^{a} | Other known affinity | humans (h) | Reference^{b} |
| Benzomorphans | | | | |
| (+)-N-allyl-normetazocine, (+)-SKF-10,047 | +++, | Kᵢ = 0.6 μM NMDAR, | (h), (a) | 1, 4 |
| | Kᵢ = 58 nM | Kᵢ = 14 μM sigma 2 | | |
| (+)-pentazocine | +++, | No | (h), (a) | 1 |
| | K_{d} = 3 nM | | | |
| (+)-3-PPP, R(+)-3-(3-hydroxyphenyl)-N- | +++, | Dopamine receptor | | 1, 5 |
| propylpiperidine | K_{d} = 10 nM | | | |
| Antidepressant | | | | |
| Imipramine | ++ | MAOI | (h) | 1 |
| Fluoxetine | ++ | SSRI | (h) | 1 |
| Fluvoxamine | +++ | SSRI | (h) | 1 |
| Neurosteroid | | | | |
| Testosterone | +++/++ | | (h) | 1 |
| Pregnenolone sulphate | + | | (h) | 1 |
| PB-008, dihydroepiandrosterone sulphate | + | | (h) | 1 |
| Cocaine | + | Dopamine transporter | (h) | 1 |
| Other | | | | |
| Igmesine, JO-1784, (+) N-cyclopropylmethyl-N- | +++ | | (h) | 1 |
| methyl-1,4-diphenyl-1-ethyl-butyl-2-N | | | | |
| DTG, di-tolyl-guanidine | +++, | Sigma-2 | | 1 |
| | K_{d} = 20 nM | | | |
| SA4503, 1-(3,4-dimethoxyphenethyl)-4-(3- | +++ | No | | 1 |
| phenylpropyl) piperazine HCl | | | | |
| PRE-084 | +++ | | | 1 |
| OPC-14523 | +++ | Sigma-2, 5HT1AR | | 1 |
| Amantadine | Kᵢ = 7.4 μM | Kᵢ = X μM NMDAR | (h) | 2, 3 |
| Memantine | Kᵢ = 2.6 μM | Kᵢ = 0.5 μM NMDAR | (h) | 2, 3 |

Abbreviations:
NMDAR, N-methyl-D-aspartate receptor;
MAOI, monoamine oxidase inhibitor;
5HT1AR, serotonin 5-hydroxytryptamine 1A receptor; and
SSRI, selective serotonin reuptake inhibitor.
^{a}Inhibition constant (Kᵢ) values as indicated, or where ‘+++’ is less than 50 nM, ‘++’ is 50 nM to less than 500 nM, and ‘+’ is 500 nM to less than 10 μM.
^{b}Table references as follows: 1. Hayashi and Su. Sigma-1 receptor ligands: potential in the treatment of neuropsychiatric disorders. CNS Drugs 18(5): 269, 2004. 2. Peeters, et al. Involvement of the sigma-1 receptor in the modulation of dopaminergic transmission by amantidine. Eur. J. Neurosci. 19: 2212, 2004. 3. Kornhuber, et al. Affinity of 1-aminoadamantanes for the sigma binding site in post-mortem human frontal cortex. Neurosci. Lett. 163(2): 129, 1993. 4. Kim, et al. New morphinan derivatives with negligible psychotropic effects attenuate convulsions induced by maximal electroshock in mice. Life Sci. 72: 1883, 2003. 5. Klein and Musacchio. Hight-affinity dextromethorphan and R(+)-3-(3-hydroxyphenyl)-N-propylpiperidine binding sites in rat brain. Allosteric effects of ropizine. J. Pharmacol. Exp. Ther. 260(3): 990-9.

TABLE III
Sigma-1 Antagonists.

| | | | Used in | |
|---|---|---|---|---|
| | sigma-1 | | animals (a) or | |
| Compounds | affinity^{a} | Other known affinity | humans (h) | Reference^{b} |
| Benzomorphans | | | | |
| Dextromethorphan, (+)-3-methoxy-N-methylmorphinan | Kᵢ = 205 nM, | Kᵢ = 7 μM NMDAR, | (h), | 1, 6 |
| | K_{d} = 20 nM | Kᵢ = 4-11 μM sigma-2 | antitussive | |
| Dextrorphan | Kᵢ = 144 nM | Kᵢ = 0.9 μM NMDAR, | (h), | 1 |
| | | Kᵢ = 4-11 μM sigma-2 | antitussive | |
| Dimemorfan, (+)-3-methyl-N-methylmorphinan | Kᵢ = 151 nM | Kᵢ = 17 μM NMDAR, | (h), | 1 |
| | | Kᵢ = 4-11 μM sigma-2 | antitussive | |
| 3-allyloxy-17-methylmorphinan (CPK-5) | Kᵢ = 156 nM | Kᵢ = 18 μM NMDAR, | | 5 |
| | | Kᵢ = 14 μM sigma 2 | | |
| 3-cyclopropylmethoxy-17-methylmorphinan (CPK-6) | Kᵢ = 148 nM | Kᵢ = 18 μM NMDAR, | | 5 |
| | | Kᵢ = 15 μM sigma-2 | | |
| Neurosteroid | | | | |
| Progesterone | +++/++ | | (h) | 2 |
| Antipsychotic | | | | |
| Haloperidol | Kᵢ = 32 nM | Kᵢ = 48 μM NMDAR, | (h), | 2, 5 |
| | | Kᵢ = 0.189 μM sigma-2 | antipsychotic | |
| Other | | | | |
| BD-1047, [2-(3,4-dichlorophenyl)ethyl]-N-methyl-2- | | | | 3 |
| (diamino)ethylamine | | | | |
| BD-1063, 1(−)[2-(3,4-dichlorophenyl)ethyl]-4-methylpiperazine | | | | 3 |
| BD-1008, N-[2-(3,4-dicholophenyl)ethyl]-N-methyl-2-(1- | +++ | sigma-2 | | 2 |
| pyrrolidinyl)ethylamine | | | | |
| NE-100, N,N-dipropyl-2-[4-methoxy-3-(2-phenylethoxy)phenyl] | +++ | | | 2, 4 |
| ethylamine HCl | | | | |
| XJ448, 1-(cyclopropylmethyl)-4-(2′,4′-cianophenyl)-2′-oxoethyl)- | ? | | | 4 |
| piperidine HBr | | | | |
| Dup-734, 1-(cyclopropylmethyl)-4-(2′-oxoethyl) piperidine HBr | +++ | No | | 2, 4 |
| MS-377, (R)-(+)-1-(4-chlorophenyl)-3-[4-(2- | +++ | No | | 2 |
| methoxyethyl)piperazin-1-yl] methyl-2-pyrrolidinone L-tartrate | | | | |
| Rimcazole, BW-234U | +, Kᵢ = 3 μM | | (h) | 2 |
| BMS-181100, BMY-14802 | ++ | | (h) | 2 |
| Panamesin, EMD-57445 | +++ | | (h) | 2 |

Abbreviations:
NMDAR, N-methyl-D-aspartate receptor.
^{a}Inhibition constant (Kᵢ) values as indicated, or where ‘+++’ is less than 50 nM, ‘++’ is 50 nM to less than 500 nM, and ‘+’ is 500 nM to less than 10 μM.
^{b}Table references as follows: 1. Chou, et al. Binding of dimemorfan to sigma-1 receptor and its anticonvulsant and locomotor effects in mice, compared with dextromethorphan and dextrorphan. Brain Res. 821(2): 516. 2. Hayashi and Su. Sigma-1 receptor ligands: potential in the treatment of neuropsychiatric disorders. CNS Drugs 18(5): 269, 2004. 3. Matsumoto, et al. Characterization of two novel sigma receptor ligands: antidystonic effects in rats suggest sigma receptor antagonism. Eur. J. Pharmacol. 14: 280(3), 301, 1995. 4. Chaki, et al. Regulation of NMDA-induced [3H]dopamine release from rat hippocampal slices through sigma-1 binding sites. Neurochem. Int. 33(1): 29, 1998. 5. Kim, et al. New morphinan derivatives with negligible psychotropic effects attenuate convulsions induced by maximal electroshock in mice. Life Sci. 72: 1883, 2003. 6. Klein and Musacchio. Hight-affinity dextromethorphan and R(+)-3-(3-hydroxyphenyl)-N-propylpiperidine binding sites in rat brain. Allosteric effects of ropizine. J. Pharmacol. Exp. Ther. 260(3): 990-9.

TABLE IV
Sigma-1 ligands with undefined agonist/antagonist activity.

| | sigma-1 | | Used in animals (a) | |
|---|---|---|---|---|
| Compounds | affinity | Other known affinity | or humans (h) | Reference^{b} |
| (−)-PPAP, R(−)-N-(3-phenyl-1-propyl)-1-phenyl-2- | +++ | NMDAR | | 1 |
| aminopropane | | | | |
| 2-[4-(4-methoxy-benzyl)piperazin-1-yl-methyl]4-oxo[4H]- | +++ | No | (a), potentiated | 2 |
| benzo-thiazolin-2-one, S-21377 | | | NMDA-induced | |
| | | | neuronal activation, | |
| | | | presumptive agonist | |
| 2[(4-benzyl piperazin-1-yl) mothyl] naphthalene, | +++ | No | (a), potentiated | 2 |
| dichiorydrate (S-21378) | | | NMDA-induced | |
| | | | neuronal activation, | |
| | | | presumptive agonist | |
| Arylakyl 4-benzyl piperzine derivatives | + to +++ | Various to 5HT1A and | (a), presumptive | 3 |
| | | D2 receptors | agonists | |
| (4-phenylpiperidinyl)- and (4-phenylpiperazinyl)alkyl spaced | + to +++ | No | (a), | 4 |
| esters of 1-phenylcyclopentanecarboxylic acid | | | | |
| Eliprodil, SL 82.0715 | +++ | NMDAR polyamine site | (h), anti-ischemic | 5 |
| Ifenprodil | | | (h), vasodilator | 6 |
| Trifluperidol | | | (h), antipsychotic | 6 |
| 4-phenyl-1-(4-phenylbutyl)-piperidine | | | | 6 |
| SR-31747A | +++ | C8-C7 sterol isomerase | | 5 |
| NPC 16377, 6-[6-(4-hydroxypiperidinyl)hexyloxy]-3- | +++ | No | blocks locomoter | 7 |
| methylflavone | | | activity stimulated | |
| | | | by cocaine, | |
| | | | presumptive | |
| | | | antagonist | |
| Carbetapentane | +++, IC₅₀ = | | (h), antitussive | 8 |
| | 9 nM | | | |
| Caramiphen | +++, IC₅₀ = | High-affinity for M1 | (h), antitussive | 8 |
| | 25 nM | muscarinic receptor | | |
| Dimethoxanate | +++, IC₅₀ = | — | (h), antitussive | 8 |
| | 41 nM | | | |
| Pipazethate | +++, IC₅₀ = | | (h), antitussive | 8 |
| | 190 nM | | | |
| Fragmented dextromethorphan | + to +++ | | | 9 |

Abbreviations:
NMDAR, N-methyl-D-aspartate receptor; and
5HT1AR, serotonin 5-hydroxytryptamine 1A receptor.
^{a}Inhibition constant (Kᵢ) values as indicated, or where ‘+++’ is less than 50 nM, ‘++’ is 50 nM to less than 500 nM, and ‘+’ is 500 nM to less than 10 μM.
^{b}Table references as follows: 1. Nishikawa, et al. Involvement of direct inhibition of NMDA receptors in the effect of sigma-receptor ligands on glutamate neurotoxicity in vitro. Eur. J. Pharmacol. 404(1-2): 41, 2000. 2. Gronier and Debonnel. Involvement of sigma receptors in the modulation of the glutamatergic/NMDA neurotransmission in the dopaminergic systems. Eur. J. Pharmacol. 368(2-3): 183, 1999. 3. Younes, et al. Synthesis and structure-activity relationships of novel arylalkyl 4-benzyl piperazine derivatives as sigma site selective ligands. Eur. J. Med. Chem. 35(1): 107, 2000. 4. Hudkins, et al. Novel (4-phenylpiperidinyl)- and (4-phenylpiperazinyl)alkyl-spaced esters of 1-phenylcyclopentanecarboxylic acids as potent sigma-selective compounds. J. Med. Chem. 37(13): 1964. 5. Hayashi and Su. Sigma-1 receptor ligands: potential in the treatment of neuropsychiatrica disorders. CNS Drugs 18(5): 269, 2004. 6. Whittemore, et al. Antagonism of N-methyl-D-aspartate receptors by sigma site ligands: potency, subtype-selectivity and mechanisms of inhibition. J. Pharmacol. Exp. Ther. 282(1): 326. 7. Witkin, et al. Effects of the selective sigma receptor ligand, 6-[6-(4-hydroxypiperidinyl)hexyloxy]-3-methylflavone (NPC 16377), on behavioral and toxic effects of cocaine. J. Pharmacol. Exp. Ther. 266(2): 473, 1993. 8. Craviso and Musacchio. High-affinity dextromethorphan binding sites in guinea pig brain. II. Competition experiments. Mol. Pharmacol. 23(3): 629, 1983. 9. Arrington, et al. Synthesis of potent sigma-1 receptor ligands via fragmentation of dextromethorphan. Bioorg. Med. Chem. Lett. 14(7): 1807, 2004.

TABLE V
Patients with AF-NT treated and not treated with the NMDA antagonist dextromethorphan.

| | | | | | | | | | Time | |
|---|---|---|---|---|---|---|---|---|---|---|
| | | | | | | | | Time to | to resolu- | Grade |
| | | | | | Time | | | initial | tion of | (1-3) of |
| Ex- | | | | | Since | | | re- | all | symptom |
| am- | Diag- | | | | MTX | | | sponse | symptoms | resolu- |
| ple | nosis | Age | Sex | MTX Therapy | (days) | Physical Findings | Oral DM Dose | (hours)\hat{ } | (hours)\hat{ } | tion* |
| 1 | NHL | 32 | M | 12 mg IT | 2 | Headache, dysarthria, | 1 mg/kg | 3 | 24 | 3 |
| | | | | | | nausea, weakness, asthenia | | | | |
| 2 | ALL | 15 | M | 12 mg IT; | 7 | Left hemiparesis | 2 mg/kg × 1 | 0.50 | 6 | 3 |
| | | | | 100 mg/m² IV | | | | | | |
| 3 | ALL | 19 | M | 7.5 mg IO weekly; | 12 | Right CN VII palsy, right | 1 mg/kg TID | 3 | 240 | 1 |
| | | | | 1 g/m² IV | | hemiparesis, dysarthria | | | | |
| 4 | OS | 13 | M | 12 g/m² IV | 7 | Right CN VII palsy, left | 1 mg/kg TID | 0.75 | 72 | 2 |
| | | | | | | hemiparesis, dysarthria, | | | | |
| | | | | | | impaired gag | | | | |
| 5 | OS | 16 | M | 12 g/m² IV | 7 | Dysarthria, CN VII palsy | 1 mg/kg × 1 | 0.50 | 0.50 | 3 |
| 6 | ALL | 18 | F | 12 mg IT | 4 | Disorientation, rambling | 1 mg/kg BID | 24 | 192 | 2 |
| | | | | | | slurred speech | | | | |
| 7 | ALL | 6 | F | 12 mg IT | 5 | Right CN VII palsy, paresis | 1 mg/kg BID × 3 | 4 | 4 | 3 |
| | | | | | | of right upper extremity | | | | |
| 8 | ALL | 9 | F | 12 mg IT | 4 | Headache, dysarthria, drooling, | 1 mg/kg BID | 2 | 48 | 2 |
| | | | | | | left arm weakness, ataxia | | | | |
| 9 | ALL | 14 | M | IV and IT MTX | 12 | Tonic colonic seizure | 1 mg/kg TID | 2 | 3 | 3 |
| 10 | ALL | 8 | F | 12 mg IT | 2 | Roving hemiparesis, more on right | 1 mg/kg BID | 36 | 120 | 2 |
| 11 | ALL | 15 | F | 12 mg IT | 4 | Right sided weakness | 1 mg/kg BID | 24 | 192 | 3 |
| 12 | ALL | 5 | M | IV and IT MTX | 8 | Seizure, dysconjugate gaze | 1 mg/kg TID | 24 | 24 | 3 |
| 13 | ALL | 13 | M | 12 mg IT | 3 | Disoriented (possibly post-ictal) | 1 mg/kg BID | 24 | 24 | 3 |
| 14 | ALL | 14 | F | 12 mg IT | 7 | Left CN VII palsy, decreased | 1 mg/kg BID | 216 | >1000 | 1 |
| | | | | | | consciousness, slurred speech | | | | |
| 15 | ALL | 13 | F | 12 mg IT | 9 | Right hemiparesis, difficulty | 1.5 mg/kg BID × 6 | — | >1000 | 1 |
| | | | | | | swallowing/talking, agitated | | | | |
| 16 | ALL | 6 | M | 12 mg IT | 2 | Dysmetria, ataxia, right upper | 1 mg/kg BID × 5 | 24 | 120 | 2 |
| | | | | | | extremity weakness | | | | |
| 17¹ | NHL | 32 | M | 12 mg IT | 2 | Headache, dysarthria, nausea, | — | — | 336 | 1 |
| | | | | | | weakness, asthenia | | | | |
| 18² | ALL | 12 | M | 15 mg/m² IT | 11 | Dysarthria, left hemiparesis | — | 48 | 336 | 1 |
| 19² | ALL | 5 | F | 15 mg/m² IT | 14 | Right CN VII palsy, slurred | — | — | 144 | 1 |
| | | | | | | speech, aphasia, right hemi- | | | | |
| | | | | | | paresis | | | | |
| 20² | ALL | 3 | M | 15 mg/m² IT | 10 | Right CN VII palsy, right hemi- | — | 4 | 144 | 1 |
| | | | | | | paresis, drowsy | | | | |
| 21³ | OS | 6 | F | 12 g/m² IV | 5 | Trance-like, tonic-colonic | — | — | 72 | 2 |
| | | | | | | seizure, left hemiparesis | | | | |
| 22³ | OS | 18 | F | 12 g/m² IV | 6 | Left hemiparesis, aphasia | — | — | 120 | 1 |
| 23⁴ | OS | ~12 | — | 12 g/m² IV | 5 | Left CN VII palsy, sleepy, left | — | — | 240 | 1 |
| | | | | | | upper extremity paresis | | | | |
| 24⁴ | OS | ~12 | — | 12 g/m² IV | 5 | Right CN VII palsy, Broca-type | — | — | 48 | 2 |
| | | | | | | aphasia, right hemiparesis | | | | |
| 25⁴ | OS | ~12 | — | 12 g/m² IV | 16 | Aphasia, left upper extremity | — | — | 24 | 3 |
| | | | | | | weakness | | | | |
| 26⁴ | OS | ~12 | — | 12 g/m² IV | 9 | Right CN VII palsy, aphasia, | — | — | 120 | 1 |
| | | | | | | dystonia, abnormal affect, right | | | | |
| | | | | | | paresis | | | | |
| 27⁴ | OS | ~12 | — | 12 g/m² IV | 7 | Ascending neuromuscular paralysis | — | — | 168 | 1 |
| | | | | | | to bulbar area | | | | |
| 28⁴ | OS | ~12 | — | 12 g/m² IV | 8 | Headache, weakness in left arm, | — | — | 1 | 3 |
| | | | | | | slurring of speech | | | | |
| 29⁵ | ALL | 6 | M | 12 mg IT; | 2-3 | Severe headache | — | — | 168 | 1 |
| | | | | 500 mg/m² IV | | | | | | |
| 30⁵ | ALL | 3 | M | 12 mg IT; | 14 | Severe headache, nausea, vomiting | — | — | 336 | 1 |
| | | | | 500 mg/m² IV | | | | | | |

Abbreviations: MTX, methotrexate; DM, dextromethorphan (without guaifenesin); NHL, non-Hodgkins lymphoma; ALL, acute lymphoblastic leukemia; OS, osteosarcoma; M, male; F, female; IT, intrathecal; IO, intra-Ommaya; IV, intravenous; CN, cranial nerve; BID, twice daily; TID, three times a day; CT, computed tomography imaging; MRI, magnetic resonance imaging; mg, milligrams; kg, kilograms; m², body surface area in square meters.
^{\hat{ }}Does not include response time to arresting a seizure by anti-seizure medication, unless DM was the only medication given.
*1 = gradual (symptoms resolved gradually or linearly over an extended period exceeding 96 hours), 2 = moderate (majority of symptoms resolved in 25 to 96 hours), and 3 = rapid (majority of symptoms resolved in 24 hours or less).
¹Neurologic episode of patient in Example 1, untreated with DM.
²From Yim et al., Cancer 67: 2058, 1991.
³From Packer et al., Medical and Pediatric Oncology 11: 159, 1983.
⁴From Jaffe et al., Cancer 56: 1356, 1985.
⁵From Gay et al., J. Child Neurol. 4: 207, 1989, includes Ara-C as part of IT therapy.

What is claimed is:

1. A method of treating antifolate neurotoxicity in a mammal suffering from or at risk of developing antifolate neurotoxicity, comprising administering to the mammal a therapeutically effective amount of an NMDA antagonist, or a pharmaceutically acceptable salt thereof.

2. The method of claim 1, wherein the NMDA antagonist is an uncompetitive open channel blocking agent at the NMDA receptor.

3. The method of claim 1, wherein the NMDA antagonist is a sigma-1 receptor ligand.

4. The method of claim 1, wherein the NMDA antagonist is dextromethorphan.

5. The method of claim 1, wherein the NMDA antagonist is memantine.

6. The method of claim 1, wherein the NMDA antagonist is administered with an inhibitor of its own metabolism.

7. The method of claim 1, wherein the NMDA antagonist is administered parenterally, transdermally, intrathecally, or orally.

8. The method of claim 1, wherein the NMDA antagonist is administered orally as a tablet, capsule or gel capsule.

9. The method of claim 1, wherein the amount of an NMDA antagonist administered is from about 0.1 mg/kg to about 20 mg/kg.

10. The method of claim 1, wherein the amount of an NMDA antagonist administered is from about 1 mg/kg to about 5 mg/kg.

11. The method of claim 1, wherein the amount of an NMDA antagonist, or a pharmaceutically acceptable salt thereof, is administered together with a pharmaceutically acceptable carrier.

12. The method of claim 1, wherein the antifolate neurotoxicity is caused by an antifolate selected from the group consisting of methotrexate, aminopterin, trimetrexate, edatrexate, raltritrexed and lometrexol.

13. The method of claim 1, wherein the NMDA antagonist is administered before, after, or simultaneously with an antifolate.

14. The method of claim 1, comprising the additional step or steps of administering one or more other compounds selected from the group consisting of leucovorin, S-adenosyl-methionine, betaine, vitamin B₆, vitamin B₁₂, folic acid, aminophylline, tetrahydrobiopterin, L-dopa, carbidopa, 5-hydroxytryptophan, and a second NMDA antagonist.

15. A dosage form comprising an NMDA antagonist, wherein the dosage form directs a patient to take a dosage of antifolate and a dosage of an NMDA antagonist, wherein the dosage duration of the NMDA antagonist is shorter than the efficacy duration of the antifolate.

16. A dosage form according to claim 15, wherein the dosage form further directs a patient to take a plurality of NMDA antagonist doses over a first continuous period and not over a second continuous period, and wherein the first and second continuous periods alternately repeat on a weekly cycle and the second continuous period is 4 to 6 days in length.

17. A combination dosage form comprising an antifolate and an NMDA antagonist.

18. The combination dosage form of claim 17, wherein the antifolate and NMDA antagonist are in intimate admixture with one another in a single dosage form.

19. The combination dosage form of claim 17, wherein the antifolate and NMDA antagonist are in a multiple dosage form.

20. The combination dosage form of claim 17, wherein the dosage duration of the NMDA antagonist is shorter than the duration of antifolate efficacy.

21. The combination dosage form of claim 17, wherein the antifolate and NMDA antagonist are each administered weekly in a multiple dosage form that directs a patient to take a plurality of NMDA antagonist doses over a first continuous period and not over a second continuous period, and wherein the first and second continuous periods alternately repeat on a weekly cycle and the second continuous period is 4 to 6 days in length.

22. The combination dosage form of claim 17, wherein the NMDA antagonist is selected from the group consisting of dextromethorphan, dextrorphan, amantadine, memantine and pharmaceutically acceptable salts thereof.

23. The combination dosage form of claim 17, wherein the dosage form is sufficient to treat a mammal suffering from or at risk of developing antifolate neurotoxicity.

24. A multiple dosing form package for sequential weekly oral administration of antifolate and NMDA antagonist tablets comprising:
a. a carrier sheet provided with one or more substantially duplicate groups of compartments, each group of compartments arranged in substantially parallel rows of one to a plurality of substantially evenly spaced compartments,
b. a first row having an NMDA antagonist tablet in each compartment,
c. a second row having one or more antifolate tablets in each compartment, where one or more compartments in the second row are aligned in one or more columns with one or more compartments in the first row,
d. a pressure rupturable cover over each of the compartments,
whereby tablets in each group of compartments represent a week supply of antifolate and NMDA antagonist suitable for treating antifolate neurotoxicity, and tablets in the first and second row compartments that lie in the same column are to be taken by the patient at substantially the same time.

25. The multiple dosing form package of claim 24, further comprising a third row having a folic acid tablet in each compartment, where one or more compartments in the third row are aligned in one or more columns with one or more compartments in the first and second rows.

26. The multiple dosing form package of claim 24, further comprising lines of severability between the groupings, thereby allowing a week supply of antifolate and NMDA antagonist to be conveniently separated from the package.

27. The multiple dosing form package of claim 24, wherein the carrier sheet and the cover are sandwiched between a pair of apertured panels, each compartment being received within an aperture of one of the panels, adjacent apertures being in substantial registry with one another.

28. The multiple dosing form package of claim 24 having daily, time, and weekly indicia beneath each of the one or more substantially duplicate groups of compartments.

29. The multiple dosing form package of claim 24 having antifolate and NMDA antagonist indicia aside each of the substantially parallel rows.

30. The multiple dosing form package of claim 24, wherein the carrier sheet is transparent and the cover material is tin foil.

31. The multiple dosing form package of claim 24, wherein tablets in each group of compartments represent a week supply of antifolate and NMDA antagonist suitable for treating antifolate neurotoxicity for a full week, and wherein the NMDA antagonist tablets are to be taken by the patient over a period of 6 days or less.
